SEQUENCE LISTING (8 sequences)

SEQ ID NO: 1 - protein, 8 aa, Artificial Sequence
  chemically synthesized peptide
KAHKKRAD

SEQ ID NO: 2 - protein, 8 aa, Artificial Sequence
  chemically synthesized peptide
KARKKHAD

SEQ ID NO: 3 - protein, 4 aa, Artificial Sequence
  chemically synthesized peptide
HKKR

SEQ ID NO: 4 - protein, 4 aa, Artificial Sequence
  chemically synthesized peptide
RKKH

SEQ ID NO: 5 - protein, 5 aa, Artificial Sequence
  chemically synthesized peptide
RSECD

SEQ ID NO: 6 - protein, 11 aa, Artificial Sequence
  chemically synthesized peptide
CTRKKHDNAQC

SEQ ID NO: 7 - protein, 8 aa, Artificial Sequence
  chemically synthesized peptide
CTRKKHDC

SEQ ID NO: 8 - protein, 8 aa, Artificial Sequence
  chemically synthesized peptide
CARKKHDC

(19) United States
(12) Patent Application Publication
(10) Pub. No.: US 2013/0149245 A1
(43) Pub. Date: Jun. 13, 2013

(54) Novel Peptides and Uses Thereof

(71) Applicants: UNIVERSITY OF SOUTHERN CALIFORNIA, Los Angeles, CA (US); CHILDREN'S HOSPITAL LOS ANGLES, Los Angeles, CA (US)

(72) Inventors: Mehran Neshat, Los Angeles, CA (US); Francis Markland, Manhattan Beach, CA (US); Rex Moats, Pasadena, CA (US)

(73) Assignees: CHILDREN'S HOSPITAL LOS ANGELES, Los Angeles, CA (US); UNIVERSITY OF SOUTHERN CALIFORNIA, Los Angeles, CA (US)

(21) Appl. No.: 13/623,796

(22) Filed: Sep. 20, 2012

Related U.S. Application Data
(63) Continuation of application No. 12/738,619, filed on Apr. 16, 2010, now abandoned.
(60) Provisional application No. 60/980,705, filed on Oct. 17, 2007.

(51) Int. Cl. A61K 49/00; G01N 33/574

(52) U.S. Cl. CPC A61K 49/00; G01N 33/57492
USPC 424/9.1; 530/328; 530/327; 530/326; 530/325; 530/324; 514/21.7; 514/21.6; 514/21.5; 514/21.4; 514/21.3; 435/375; 435/7.23

(57) ABSTRACT

The invention relates to a peptide of 8-50 amino acids comprising the sequence of KAHKKRAD or KARKKHAD, or a cyclic peptide of 8-50 amino acids comprising the sequence of HKKR or RKKH. Also disclosed are methods of using the peptide for detecting, monitoring, or treating cancer.

### RELATED APPLICATION

This application claims priority to U.S. Provisional Application Ser. No. 60/980,705, filed on Oct. 17, 2007, the content of which is incorporated herein by reference in its entirety.

### FIELD OF THE INVENTION

The present invention relates primarily to cancer and other pathologies dependent on the activity state of ligands of described peptides. More specifically, the invention relates to peptides having the sequence of KAHKKRAD or KARKKHAD in cyclic or linear form and cyclic peptides having the sequence of HKKR or RKKH, as well as use of the peptides for detecting, monitoring, and treating cancer.

### BACKGROUND OF THE INVENTION

Cancer is a heterogeneous disease at the individual and population level. Interaction of cancer cells with their microenvironment involves intra- and extra-cellular molecular components in which critical pathways may differ among patients, cellular constituents, and progressive stages of the disease. Consequently, effective targeted therapy requires definition of molecularly defined disease subtypes based on:

i) Identification of indispensable biological functions that critical cellular components rely on.

ii) Identification of molecules that mediate these effects among nodes amenable to molecular intervention.

Successful testing and application of such directed therapies is further dependent on definition of patient populations in which characterized molecular mechanisms are in effect. This objective requires:

i) Development of modalities that measures abundance, localization and activity state of these molecular targets in longitudinal studies during the course of disease progression.

ii) Evaluation of safety, efficacy and specificity in preclinical models and translation in clinical trials.

Within the last decade, this paradigm has been applied and proven effective in multiple forms of cancer⁽¹⁾. Breast cancer is among the first in which causal determinants of the disease have been incorporated into directed therapeutic interventions^{(2, 3)}. In addition, classification of breast cancer subtypes is now extended to the transcriptome profiles of primary cancer cells^{(4, 5)}. However, while crucial, current molecular targets in multiple forms of cancers are incomplete, restricted to primary cancer cells, and lack necessary non-invasive diagnostic tools for clinical applicability.

### SUMMARY OF THE INVENTION

The present invention is based, at least in part, upon the unexpected discovery that peptides having the sequence of KAHKKRAD or KARKKHAD and cyclic peptides having the sequence of HKKR or RKKH can be used to detect, monitor, and treat cancer.

Accordingly, in one aspect, the invention features a linear or cyclic peptide comprising the sequence of KAHKKRAD or KARKKHAD, or a cyclic peptide comprising the sequence of HKKR or RKKH. The length of the peptide is in the range of 8-50, 8-20, or 8-12 amino acids.

The peptide may be cyclized via a link between a side chain and the backbone, or alternatively, via a link between two reactive groups on the backbone. For example, the peptide may be cyclized via a link between the side chain of D and the backbone of K. The peptides may be in monomeric or multimeric form.

In some embodiments, the peptide is detectably labeled. In some embodiments, the peptide is linked to another molecule such as an imaging or therapeutic agent. The linkage may be through linkers that can be modified by the biological processes of the target cell.

Another aspect of the invention relates to a composition comprising a pharmaceutically acceptable carrier and a peptide of the invention.

The invention further provides a method of binding a peptide of the invention to an aI domain. The method comprises contacting the peptide with the aI domain, thereby allowing binding of the peptide to the aI domain.

In some embodiments, the aI domain is in α₂, α₁, α₁₀, or α₁₁. In particular, the aI domain may be in α₂β₁. The aT domain may be on or in a cell such as a cancer cell (e.g., a breast or ovarian cancer cell). In some embodiments, the cell is in a subject such as a mouse.

Also within the invention is a method of detecting cells expressing an aI domain in an open ligand binding conformation. The method comprises contacting a peptide of the invention with a cell and detecting binding of the peptide to an aI domain on or in the cell.

In some embodiments, the cell is a cancer cell, e.g., a breast or ovarian cancer cell. In some embodiments, the cell is in a subject such as a mouse. The method may further comprise isolating the cell that binds the peptide, which may be a cancer cell or cell from the subject.

The binding of the peptide to the aI domain may be detected by imaging. In some embodiments, the binding of the peptide to the aI domain is detected by detecting the peptide on or in the cell. The binding of the peptide to the aI domain or additional targets, if at a level higher than that for a normal control cell, indicates that the cell is a cancer cell or contributes to cancer progression.

In addition, the invention features a method of modulating the biological function or localization of a molecule having an aI domain. The method comprises contacting a peptide of the invention with a molecule having an aI domain, thereby modulating the biological function or localization of the molecule.

The molecule may be on or in a cell. In some embodiments, the cell is a cancer cell, e.g., a breast or ovarian cancer cell. In some embodiments, the cell is in a subject such as a mouse.

Moreover, the invention provides a method of monitoring cancer status in a subject. The method comprises introducing cancer cells into a subject, allowing the cancer to progress at the primary site or to metastasis in the subject, administering a peptide of the invention to the subject, and detecting the peptide on or in the cancer cells, thereby monitoring the status of the cancer in the subject.

In some embodiments, the subject is mouse. The cancer may be breast or ovarian cancer. In some embodiments, the peptide is detected by imaging.

Additionally, a cell of subline MDA-MB-231-MBF1C-Luc, MDA-MB-231-MBF1C-Luc-GFP, or MDA-MB-231-MM-Luc is within the invention.

In yet another aspect, the invention provides a method of monitoring cancer status in a subject. The method comprises administering a peptide of the invention to a subject having cancer cells and detecting the peptide on or in the cancer cells, thereby monitoring the status of the cancer in the subject.

Unless otherwise defined, all technical and scientific terms used herein have the same meaning as commonly understood by one of ordinary skill in the art to which this invention pertains. In case of conflict, the present document, including definitions, will control. The materials, methods, and examples disclosed herein are illustrative only and not intended to be limiting. Other features, objects, and advantages of the invention will be apparent from the description and the accompanying drawings, and from the claims.

### BRIEF DESCRIPTION OF THE FIGURES

FIG. 1. Targeted Imaging.

FIG. 2. Breast Cancer Cell Lines.

FIG. 3, Differential Integrin Profile in MDA-MB-231 Derived Sublines.

FIG. 4. Targeted Imaging of Integrins.

FIG. 5. Cellular Models: Breast Cancer MDA-MB-231 Sublines.

FIG. 6. Characterization of MDA-MB-231 Sublines.

FIG. 7. Activity-Based Imaging.

FIG. 8. In Vitro Binding Profile of Active α₂β₁ Reactive Peptides.

FIG. 9. In Vivo Fluorescent Imaging of Active α₂β₁-Reactive Peptides.

FIG. 10. aI Domain Targeted Peptides: In Vitro Binding.

FIG. 11. aI Domain Targeted Peptides: in Vitro Specificity.

FIG. 12. In Vivo Functional Imaging: Early Tissue Distribution and Tumor Targeting—Breast Cancer Model.

FIG. 13. In Vivo Ovarian Cancer Models: Intra-Peritoneal Implants—Reproductive Organs.

FIG. 14. In Vivo Ovarian Cancer Models: Intra-Peritoneal Implants—Metastases.

FIG. 15. In Vivo Ovarian Models: Orthotopic Implants.

FIG. 16. Longitudinal optical imaging of MDA-MB-231-Luc-MBF1C (α₂β₁ hi) xenografts: (A) SCID middle aged female mice were implanted with 10⁴ or 10⁶ MDA-MB-231-Luc-MBF1C cells from in vitro cultures in the indicated mammary fat pads. Developed tumors were imaged by Xenogen optical imaging after systemic luciferin administration via tail vein of anesthetized animals. (B) Photon flux was measured longitudinally in indicated areas during the course of tumor growth.

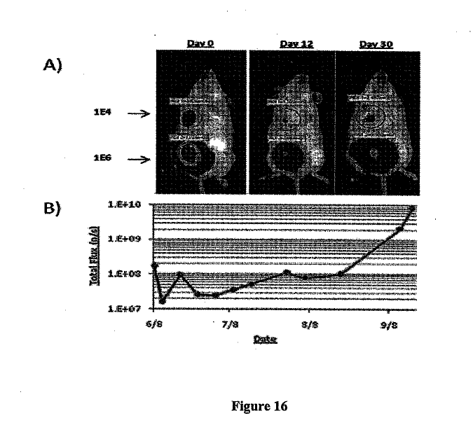

FIG. 17. Confocal Image of Peptide 1 in Glucose-Deprived OVCAR-3 Cells on Collagen 1 Matrix.

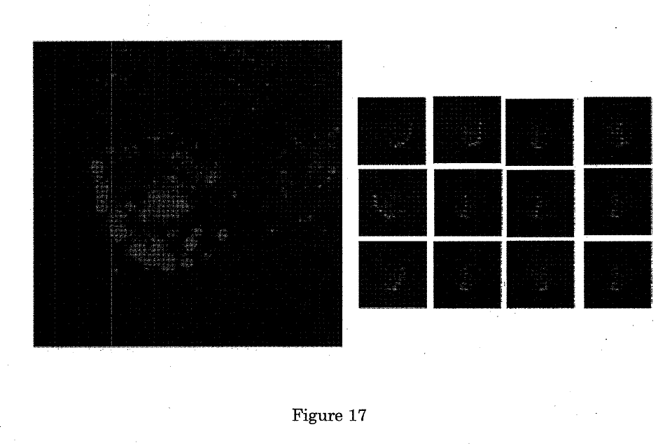

FIG. 18. Confocal Image of Peptide 1 in Glucose-Deprived OVCAR-3 Cells in Suspension Cultures.

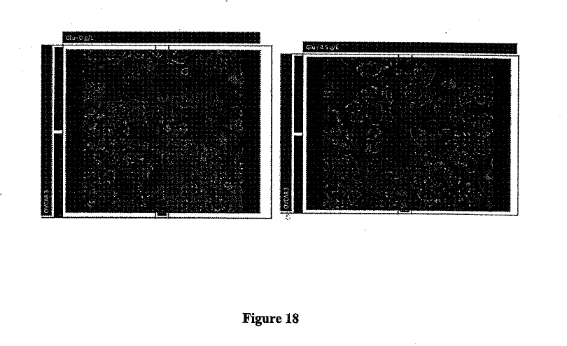

FIG. 19. Effects of Rapamycin on In Vitro Binding and Uptake of Peptide 1 in A2780 cells.

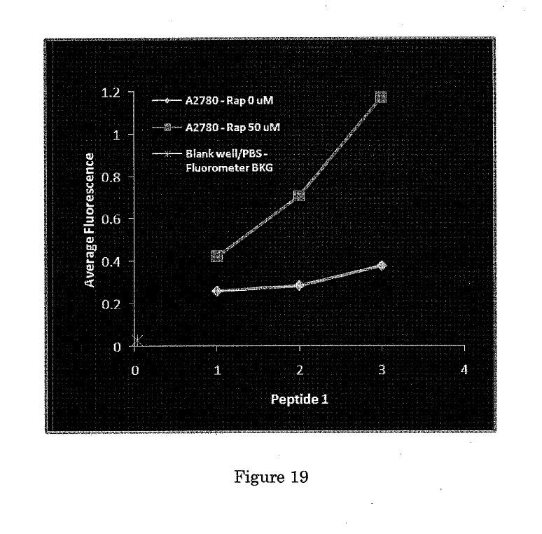

FIG. 20. Tissue Distribution and Clearance Kinetics of I.V.-Administered Peptide 1 in Live Young Female Nu/Nu Mice as Imaged by Xenogen Biofluorescent Imaging.

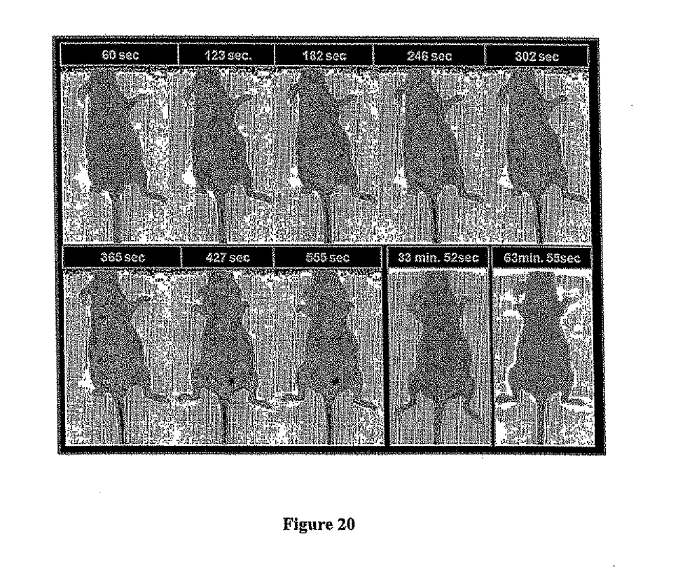

FIG. 21. Early Distribution of I.P.-Administered Peptide 1 in Female Nu/Nu Mice with Intra-peritoneal Xenografts of the A2780 Ovarian Cancer Cells.

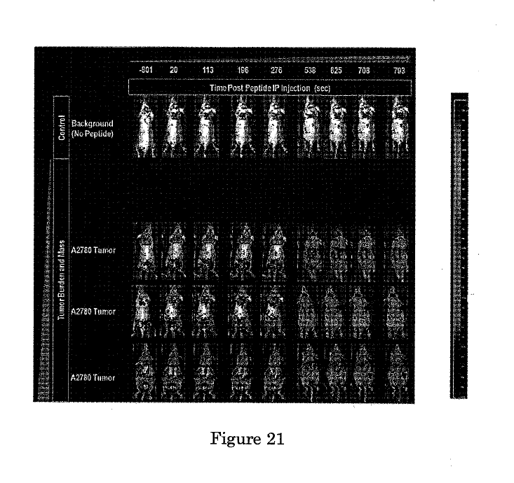

### DETAILED DESCRIPTION OF THE INVENTION

Current markers of breast cancer subtypes are restricted to molecular expression in correlative analyses, as opposed to functional and biological approaches. In contrast, a functional approach has been taken in this application toward the aim of development of reagents that can specifically recognize the activity state of a subset of integrins and molecules that regulate the activity state of dependent pathways. In turn developed reagents allow non-invasive imaging of the activity state of respective ligands at the cellular and organism level. Furthermore, the therapeutic potential of modulation of biological processes associated with the imaged active receptor is examined.

Integrin function has been proven indispensable for cancer progression. Toward further definition of critical nodes essential to specific subtypes of cancers, a section of the studies has examined the role of active form of α₂β₁ integrin as a member of this group of receptors whose activity state is associated with structural changes exposing the aI domain. Several lines of evidence point to the functional importance of α₂β₁ in the biology of breast cancer and as a prime candidate for targeted intervention:

i) In breast cancer, polymorphism in the α₂ gene is associated with progression risk⁽⁶⁻⁸⁾.

ii) α₂β₁ is a drugable target since genetic knock out of α₂ is tolerated in mice⁽⁹⁻¹¹⁾.

iii) Structural features of α₂β₁ allow design of high avidity domain specific ligands specific to the activity status of the receptor. Specifically, α₂ is among few integrins whose activation is accompanied by conformational change exposing the aI domain, as well as clustering in microdomains at the cell surface⁽¹²⁾.

iv) Interaction of cancer cells with extracellular matrix is important to cancer metastasis⁽¹³⁻¹⁵⁾. α₂β₁ is a major collagen and laminin receptor in a cell type specific manner⁽¹⁶⁻¹⁷⁾. Modulation of α₂β₁ expression and function by interacting proteases highly expressed in osteoclast, bone, and lung has been suggested⁽¹⁸⁻¹⁹⁾. These tissues constitute preferential sites of metastasis in advanced breast cancer.

v) Cross regulation of α₂β₁ expression and function by growth factor receptors is indicative of the role of α₂β₁ in defining the context for pro-growth/-survival instructive extracellular signals. EGF family of receptors is proven to direct breast cancer initiation and progression. In this respect, expression, membrane localization and internalization of α₂β₁ are regulated by EGF and ErbB2⁽²⁰⁻²²⁾. Conversely, α₂β₁ dependent regulation of VEGF, a pro-survival and angiogenic growth factor has been reported⁽²³⁻²⁴⁾.

vi) Pathways mediating transduction of signal downstream of α₂β₁ are well studied. α₂β₁-dependent pathways, such as PI3K and MAPK, regulate multiple cellular survival mechanisms⁽²⁵⁻²⁷⁾. Among them, autophagy is important in development of breast and tissue remodeling during pregnancy⁽²⁸⁻²⁹⁾. Significantly, autophagy is critical to development and progression of breast cancer⁽³⁰⁻³⁶⁾. Haplo-insufficiency of beclinl, a mediator of autophagy, leads to breast cancer development in engineered murine models^{(37, 38)}. Furthermore, prolonged autophagic survival can lead to differential response to DNA damage and has been postulated to promote genetic instability^{(39, 40)}. Several adhesion molecules have been shown to modulate autophagy⁽⁴¹⁻⁴⁴⁾. In addition, autophagy modulates the organization of cytoskeletal filaments and promotes cell survival after cell detachment from extracellular matrix⁽⁴⁵⁻⁴⁸⁾.

vii) The role of α₂β₁ is well described in thrombosis⁽⁴⁹⁾, inflammation⁽⁹⁾, angiogenesis⁽⁹⁾ and wound healing^{(10, 51)}. In respect to angiogenesis, xenografts of human breast cancer cell lines in α₂β₁ null mice reveals differential tumor vascularization dependent on the molecular expression profile of primary cancer cells and integrin status in host derived cells⁽²³⁾. In addition, angiogenic inhibitors such as endostatin, a proteolytic fragment of collagen, similarly induce autophagy^{(52, 53)}.

viii) Breast is a hormone-dependent tissue and hormone receptor status defines the biology and progression stage of breast cancer. Accordingly, α₂β₁ is hormonally regulated, predominantly localized to terminal ductal epithelia, and involved in its differentiation and branching⁽⁵⁴⁻⁶¹⁾.

ix) Existence of multipotent transplantable progenitor populations in multiple forms of cancers is documented. Importantly, α₂β₁ defines distinct population of progenitors in breast, prostate, colon, liver and bone marrow⁽⁶²⁻⁷¹⁾.

Furthermore, in ovarian cancers:

i) Primary tumors, associated endothelial lining, and ovarian cancer cell lines have been shown to differentially utilize α₂β₁ integrin as compared to normal tissue. Specifically, level of α₂β₁ is augmented in patient's ascites in advanced stages. Similarly, in in vitro spheroids models, expression of α₂β₁ remains elevated in human ovarian cancer cell lines, as opposed to primary non malignant cells.

ii) α₂β₁ is a major collagen and laminin receptor that are critical components of mesothelial targets for ovarian cancer metastases. Furthermore, increased α₂β₁ expression correlates with and its inhibition with blocking antibody modulates expression and activation of MMP2 and MMP9.

iii) Response to conventional chemotherapeutics (taxanes) and radiation is altered in spheroids cultures and suggests that integrin-dependent caspase-independent cell death may be important.

iv) α₂β₁ cross talks and modulates other integrins such as αᵥβ₃. Similarly, cross regulation of α₂β₁ with growth factor for TGF, EGF and VEGF receptors has been well documented. Importantly, expression, membrane localization and internalization of α₂β₁ are regulated by EGF and ErbB.

Structure and Function:

Design and choice of α₂β₁ aI reactive peptides in this study has focused on the structural features of the active receptor. α₂β₁^{(OMIM 192974, GeneID/Protein: ITGA2:3673/NP}^{_{—}}^{002194, Itga2:16398/NP}^{_{—}}⁰³²⁴⁴⁾ is a heterodimeric protein and member of integrin family of surface receptors⁽⁷²⁾. The mature polypeptide chain of α₂ consists of 1152 amino acids including a transmembrane and short cytoplasmic tail. While the α chain shows limited homology to other members, cysteine residues and cation binding sites are evolutionary conserved. α₂ and α₁ are among 9 members of the α chain family of integrin whose activation is accompanied by conformational change exposing the alpha insertion (aI) domain, a 191 amino acid segment with homology to vWA domain^{(72, 12)}. Activity of α₂β₁ is further regulated by clustering in specialized microdomains at the cell surface^{(20, 73)}. The aI domain includes residues involved in ligand binding that include collagen⁽¹²⁾. Collagen and laminin are the major extracellular matrix ligands of α₂β₁, where cell type-specific differences in ligand specificity have been established^{(16, 17)}. Binding of α₂β₁ to collagen in platelets mediate activation signals dependent on src and PLCγ and is accompanied with functional and morphological changes⁽⁷⁴⁻⁷⁶⁾. Surface expression of α₂β₁ is regulated at multiple levels including transcriptional and pre-mRNA splicing mechanisms^{(6, 8, 77, 78)}. Accordingly, polymorphisms in the promoter and coding region correlate with expression density⁽⁸¹⁻⁸⁸⁾. Non-transcriptional regulation of α₂β₁ has been reported, including in its response to TPA where activity is dependent on rho-dependent mechanisms⁽⁸⁹⁻⁹¹⁾. Similarly, IFNα alters α₂β₁-dependent binding to collagen without change in its expression level^{(92, 93)}. Importantly, α₂β₁ expression and function is under hormonal control and contribute to changes in development and histology of the breast during pregnancy⁽⁵⁴⁻⁶¹⁾. Conversely, ERα has been reported to be regulated by ECM in an α₂β₁-dependent manner^{(94, 95)}.

α2 Integrin Interacting Peptides: Venom of pit viper Bethrops jararaca inhibits interaction of α₂β₁ to collagen because of the action of the jararhagin disintegrin⁽⁹⁶⁻¹⁰¹⁾. However, The RSECD sequence that replaces the conserved RGD motif in the disintegrin domain fails to inhibit collagen binding⁽¹⁰²⁻¹⁰⁴⁾. In contrast, CTRKKHDNAQC binds to aI domain and prevents its binding to collagen (type I, IV) and laminin (type 1)⁽¹⁰⁵⁻¹¹¹⁾. These findings further showed that the amino acids RKK were critical for binding, cysteines were necessary for conformational constraint, and binding was dependent on Mg²⁺ presence in the aI MIDAS domain⁽¹⁰⁶⁻¹⁰⁸⁾. Further studies confirmed that the RKKH binds the α2 aI domain near the MIDAS domain and suggest that this interaction targets the metalloproteinase to the receptor, inhibits its function and exerts proteolytic effect in proximal chains⁽⁹⁹⁾. These results are noteworthy in regard to the role of the β₁ chain of the receptor and interacting molecules within the microdomains that α₂β₁ is present in. In fact a proteolytic fragment of β₁ has been isolated upon treatment with jararhagin⁽¹¹²⁾. Other structural studies have confirmed and extended these findings and shown that CTRKKHDC and CARKKHDC peptides induce conformational change in the open conformation of α₂ receptor⁽¹⁰⁷⁾. Recombinant baculovirus expressing the RKKH motif on their surface bind peptides corresponding to the α2 aI domain, and partly aided virus entry in a PLC-independent manner⁽¹⁰⁵⁾. Fibronectin FN-C/H II peptide, a heparin binding sequence, similarly contains the cationic RKK motif. Over-expression of mutants by amino acid substitution resulted in inhibition of tumor growth in vivo independent of the mitogenic activity of the protein⁽¹¹³⁾. The sequence is also present in the PDGF B-chain loop III⁽¹¹⁴⁾. Among α₂β₁-interacting disintegrins, aggretin, a c-type lectin from the venom of Calloselasma rhodostoma, similarly activates platelets and induces angiogenesis via expression of VEGF^{(115, 116)}. In the course of the studies, other cellular proteins with HKKR or RKKH motives have been identified that have been documented to modulate integrin expression, localization and function or regulate cell survival mechanisms. Thereby, presented peptides may also function as mimetopes of these proteins. These include members of RapGAP and atg family of proteins important in integrin function and autophagic survival mechanisms.

Expression in Progenitor Populations:

Importantly, α₂β₁ is present in progenitor populations in breast, prostate, liver, colon and bone marrow⁽⁶²⁻⁷¹⁾. In the bone marrow, α₂β₁(hi) defines a later subset of hematopoietic cells that have multi-lineage capacity but reduced self renewal⁽⁶⁴⁾. In erythroid progenitors, VEGF-A down-regulates α₂ mRNA, and α₂β₁-mediated interaction with collagen alters proliferative potentials⁽⁶⁶⁾. In hormone-dependent tissue, differential progenitor potency is observed in respect to α6^{(117, 118)}. In prostate cancer cells, differential tumorogenicity is observed based on CD44 and α₂β₁ expression profile⁽⁶⁵⁾. In human neuronal stem cell, interaction with inflamed TNFα-treated endothelium is mediated by α₂⁽⁶⁹⁾. In keratinocytes, adhesion to collagen differentiates long term repopulation ability⁽⁷¹⁾. In breast, while the role of β₁ integrin and α6 are best characterized, the function of α₂β₁ in progenitor populations is less clear.

Role in Normal Physiology and Disease:

In differentiated cells, α₂β₁ is expressed on platelets, epithelial and mesenchymal cells, among others^{(Genecard GC05P052321)}. In normal differentiation, α₂β₁ is predominantly localized to terminal ductal epithelia and involved in its branching^{(55, 58)}. In addition, changes in conformation of β₁ correlate with onset of cell death in involuting glands. Population specific polymorphisms in α₂ has been documented^{(77,78, 80, 119-122)}. The role of α₂β₁ is well described in thrombosis⁽⁴⁹⁾, inflammation⁽⁹⁾, angiogenesis⁽⁹⁾ and wound healing^{(10, 51)}. In inflammation, α₂ subset of memory T cells defines a functional subclass in respect to response to intracellular bacteria^{(93, 123, 124)}. In mast cells, α₂β₁ provides a co-stimulatory response in mast cells in response to infection⁽¹²⁵⁾. Furthermore, α₂β₁ constitutes a novel receptor for collectin and C1q complement proteins⁽¹²⁶⁾. α₂β₁ has further been defined as retovirus receptor where its role is important in post-adhesion steps⁽¹²⁷⁾. In respect to angiogenesis, along with α₁β₁, tumor angiogenesis and capillary morphogenesis is regulated by endothelial α₂β₂⁽¹²⁸⁻¹³⁰⁾. α₂β₁ is up-regulated in tumor-associated microvascular endothelium⁽¹³¹⁾. In the wound healing context, deletion of α₂β₁ promotes neoangiogenesis⁽¹³²⁾. VEGF-A induces α-1 and -2, lymphatic vessel formation, and haptotactic migration^{(23, 24)}. Similarly, anti-angiogenic drug E7820 has been reported to reduce α₂β₁ expression on endothelial and platelets⁽¹³³⁾. Fragments of perlecan and thrompospondin have anti-angiogenic capacity that is dependent on α₂β₁ interactions⁽¹³⁴⁾. A dicotomy between effects of inhibitory peptides and targeted deletion of α₂β₁ in respect to angiogenesis may be due to cross talk with other tumor promoting receptors⁽²³⁾.

Role in Cancer:

Importantly, polymorphisms at residues 807 and 1648 correlate with breast cancer development risk^{(6-8, 135)}. Other polymorphisms have been linked to pathologies including thrombocytopenia⁽¹³⁶⁾ and diabetic retinopathy⁽¹³⁷⁾. In breast cancer, α₂β₁ cellular expression has been shown to be heterogeneous. In general, reduction in α₂β₁ expression has been associated with grade and progression stage^{(79, 138-141)}. Metastatic sublines with lower levels of α₂β₁ has been shown to have altered morphology and distinct ability to form 3D structures in collagen matrices^{(142, 143)}. Furthermore, re-expression of α₂β₁ has been reported in reversion of malignant phenotypes⁽¹³⁸⁾. Conversely, α₂β₁ has been shown to mediate the ability to localize and attach to cortical bone, a prominent site of breast cancer metastasis^{(19, 140, 144-148)}. Correlation of receptor with multidrug resistance has been reported as well^{(150, 151)}. Neurotransmitters such as norepinephrine, dopamine and substance P have been shown to up-regulate α₂β₁ and modulate the metastatic profile⁽¹⁵²⁾. Expression, membrane localization and internalization of α₂β₁ are regulated by EGFR that is deregulated in a large percent of breast cancer tumors⁽²⁰⁻²²⁾. Strong ErbB2 signaling has been shown to down-regulate α₂β₁⁽¹⁵³⁾. Furthermore, modulation of the receptor surface expression by EGF is dependent on caveolae raft mediated endocytosis⁽²⁰⁾. In respect to other growth factors, cross talk to PDGF in proliferating smooth muscle has been reported through a src-dependent mechanism⁽¹⁵⁴⁻¹⁵⁹⁾. Among cell surface receptors, its interaction with E-cadherin is noteworthy^{(76, 160-167)}. Loss of E-cadherin in respect to adhesion to cells and matrix is in part mediated by α₂, α₃ and β₁^{(167, 76)}. Among cross talks to other integrins, α₂β₁ re-expression has been reported to up-regulate α₆β₄^{(57, 69, 143, 151, 168)}, and its cross talk with αᵥβ₃^{(15, 131, 157, 169-178)} has been suggested to depend on MT1-MMP⁽¹⁵⁾. α₂β₁ interacting proteases, involved in tissue remodeling and growth factor signaling, are highly expressed in osteoclast, bone, heart and lung^{(18, 19)}. Interestingly, targeted deletion of α₂ in mice is not lethal and does not result in overt adverse physiology, allowing the potential to develop tolerated therapeutics against this molecule⁽⁹⁻¹¹⁾. However, α₂β₁ ablation appears to alter the angiogenic response to tumor xenografts dependent on the molecular expression profile of introduced cells⁽²³⁾.

Mechanisms of Cell Survival:

In terms of cellular survival, role of integrin in terms of anoikis- and caspase-dependent mechanisms are extensively studied^{(174, 175)}, α₂β₁ has been reported to be is involved in Fas-mediated apoptosis⁽¹⁷⁶⁾. MMP1 induced dephosphorylation of AKT and neuronal death has similarly reported to depend on mechanisms involving α₂β₁^{(139, 177)}. In breast, TRAIL-mediated apoptosis during lumen formation comprise apoptotic and autophagic components in 3D cultures⁽¹⁷⁸⁻¹⁸¹⁾. Similarly, changes in β₁ correlate with onset of apoptosis in involuting gland⁽¹⁸²⁾. Furthermore, src-mediated expression of α₂β₁ modulates integrin-dependent survival⁽⁷⁴⁾. Accordingly, ECM fragments initiate a state of resistance to apoptosis in fibroblasts via α₂β₁, src, fyn and PI3K pathways⁽¹⁸³⁾. In contrast to apoptotic cells death, mechanism of survival in progenitor populations, and extent of involvement of caspase-independent survival mechanisms in the limiting environment of tumors are not well examined. Autophagy is an evolutionary catabolic survival function in response to limiting environmental factors^{(28, 29)} and regulated by the PI3K- and mTOR-dependent pathways⁽¹⁸⁴⁻¹⁸⁹⁾. Prolonged autophagy can lead to chromosomal instability and altered cancer progression^{(40, 190, 191)}. Autophagy similarly appears to influence the necrotic vs. apoptotic decision⁽¹⁹²⁾. Prolonged autophagic states lead to type II programmed cell death in which intermediate and microfilaments are redistributed but maintained⁽⁴⁷⁾. Beclin 1, a regulator of autophagy, is monoallelically deleted in breast, prostate, and ovarian cancers^{(37, 38)}. Allelic loss of beclin 1 leads to accelerated lumen formation⁽⁸⁰⁾. BNIP3, a regulator of autophagy, is up-regulated in DCIS and invasive carcinoma of breast^{(33, 193-197)}, BNIP3 is similarly associated with increased risk and disease-free survival⁽³³⁾. Extracellular signals such as nutrient starvation, anti-estrogens or exposure to chemotherapeutics imitate autophagic mechanisms. CD166, the receptor for CD6, is an estradiol-regulated adhesion molecule that promotes survival and inhibits autophagy in breast tissue⁽⁴³⁾. In respect to other nuclear hormone receptors, EB1089, a vitamin D analog, induces autophagy^{(198, 199)}. Knowledge of the role of integrins in type II and non-caspase-dependent cell survival functions is extremely limited. In prostate cancer cells cultures on laminin, cross talk of α₃β₁ and α₆β₄ with EGFR regulate decision for apoptotic versus autophagic mechanisms⁽⁴¹⁾. In liver, RGD-based integrin interacting peptides regulate osmosensory and survival functions^{(44, 200)}.

Specific cellular components may exist within the tumor microenvironment that are critically dependent on active aI domain containing integrins. Furthermore, molecular and biological characterization of activity of this subset of integrins' function allows development of targeted diagnostic and therapeutic modalities that are differentially effective in specific cellular and patient subsets, in which these processes are indispensable to tumor progression. In these respects, the present application has at least three general objects: 1) Characterization of the biological role of active integrins expressing the aI domain toward cancer progression, and isolation of cellular populations dependent on the characterized active integrins. 2) Development of non-invasive imaging modalities that can serve for further study of the basic biology of the disease, and examine its potential for translational studies that can serve for early detection. 3) Definition of therapeutic potential of developed reagents as direct modulators of cells critically dependent on the active receptor, or as activatable targeting molecules.

Accordingly, the invention provides novel peptides for detecting, monitoring, and treating cancer. The peptides are linear or cyclic peptides comprising the sequence of KAHKKRAD or KARKKHAD, and cyclic peptides comprising the sequence of HKKR or RKKH. The length of the peptide can be anywhere in the range of 8-50, for example, 8-40, 8-30, 8-20, 8-10, 10-50, 20-40, or 30-35 amino acids. To form a cyclic peptide, the peptide may be cyclized via a link between a side chain and the backbone, or alternatively, via a link between two reactive groups on the backbone. For example, the peptide may be cyclized via a link between the side chain of D and the backbone of K.

“Cyclic peptides” refer to structurally constrained chain of amino acids that are made into structures resembling a ring or circle through linkage of parts of the molecule. Cyclization can be achieved, for instance, through disulfide bond of two side chains, amide or ester bond of two side chains, amide or ester bond of one side chain and backbone of alpha amino or carboxy groups, or amide bond of alpha amino and carboxy functional groups. Three dimensional constrained structure of the active site in cyclic peptides can thereby be made to more closely parallel the biological counterpart or better interact to potential ligands. In addition, cyclic peptides are less amenable to proteolysis and digestion and have proved to have distinct biological distribution and clearance in vivo. “Linear peptides,” in contrast, refer to chain of amino acids that are not structurally constrained through intra- or inter-molecular linkage, and are freer to adopt multiple three dimensional structures dependent on their amino acid composition and sequence.

“Backbone” and “side chain” refer to part of a peptide, where the backbone is part of the peptide that is characterized by the peptide bond creating generally a chain of alpha carbon in each amino acid, and side chain generally referring to the R group of each amino acid in the formula H₂NCHRCOOH. Cyclization through backbone to backbone refers to structural constrained conformation obtained through the covalent amide bond of the non-side chain amine and carboxylic acid functional, groups of terminal amino acids. Cyclization through side chain to backbone refers to covalent linkage of amine group of the N-terminal amino acid or the C-terminal carboxylic group with a reactive group on the side chain (R) of an amino acid in the peptide. For example, in peptides comprising the sequence of KAHKKRAD or KARKKHAD, side chain to backbone cyclization may be made through covalent linkage of the C-terminal aspartic acid side chain (R═CH₂COOH) to the non-side chain NH₂ group of N-terminal lysine.

The core recognition sequences of the peptides (HKKR or RKKH) are based on studies of jararhagin metalloproteinase disintegrin. Additional sequences surrounding the recognition motifs allow proper cyclization and potentially increase the ligand spectrum to other molecules important to integrin function and trafficking, such as RapGAPs. Constraining peptides by cyclization allows increased stability and proper three dimensional conformation. Multiple cyclization methods allow study and definition of optimal ligand binding structure.

A peptide of the invention may be detectably labeled. For example, FAM fluorescent tag may be added for detection of the molecule in preliminary in vitro and in vivo studies, and can be replaced with other moieties amenable to basic science research (optical imaging: fluorescence, bioluminescence), clinically relevant imaging modalities (MRI, PET, UltraSound: examples: metal-chelating molecules, quantum dots, other nanoparticles) and therapeutic adducts (regulator of a secondary target, novel or characterized chemo- and immunotherapeutics).

A peptide of the invention may also be linked to another molecule such as an imaging or therapeutic agent. For example, biotin moiety may be added to identify the spectrum of ligands and linkage to other molecules. The peptides are linked to biotin to allow its multimerization or non-covalent linkage to secondary molecules. The peptides can also be covalently linked to secondary molecules either directly or through a linker. Such linker can be a non-peptide, a peptide sequence containing the recognition motif of a specific peptidase, and the like.

Molecular imaging refers to visualization of molecules in living or non-living biological samples through detection of their specific interaction to molecules termed “imaging agents” that interact with the biological molecule of interest and have properties that are detectable and measurable by available or developed imaging technologies. “Activity based targeted molecular imaging” agents are here defined as imaging agents that further detect the functional activity state of the target molecule. “Therapeutic agents” refers to molecules that have benefits in stopping or management of initiation or progression of deleterious biological condition or its progression stage. “Targeted therapeutics” refers to specific modulation of function of critical molecular targets identified as indispensable to disease initiation and progression.

A peptide of the invention may be chemically synthesized or produced by a cell according to the methods well known in the art.

A peptide of the invention can be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

A peptide or composition of the invention may be used for treating cancer by administering an effective amount of a peptide of the invention to a subject suffering from cancer.

As used herein, “cancer” refers to a disease or disorder characterized by uncontrolled division of cells and the ability of these cells to spread, either by direct growth into adjacent tissue through invasion, or by implantation into distant sites by metastasis. Exemplary cancers include, but are not limited to, carcinoma, adenoma, lymphoma, leukemia, sarcoma, mesothelioma, glioma, germinoma, choriocarcinoma, prostate cancer, lung cancer, breast cancer, colorectal cancer, gastrointestinal cancer, bladder cancer, pancreatic cancer, endometrial cancer, ovarian cancer, melanoma, brain cancer, testicular cancer, kidney cancer, skin cancer, thyroid cancer, head and neck cancer, liver cancer, esophageal cancer, gastric cancer, intestinal cancer, colon cancer, rectal cancer, myeloma, neuroblastoma, and retinoblastoma.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

A subject to be treated may be identified in the judgment of the subject or a health care professional, and can be subjective (e.g., opinion) or objective (e.g., measurable by a test or diagnostic method such as those described below).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect).

A peptide of the invention may also be used to bind an aI domain in vivo and in vitro. An “aI domain” constitutes a conserved amino acid sequence present in a subset of integrins with homology to the vWF. A conformational and functional correlate exists in these integrins, in which the aI domain is exposed in the active form of the molecule. A method of binding a peptide of the invention to an aI domain comprises contacting the peptide with the aI domain, thereby allowing binding of the peptide to the aI domain. The aI domain may be contained in target molecules such as α₂, α₁, α₁₀, and α₁₁. Upon activation of the target molecules, the aI domain is exposed and becomes available for binding by the peptide. Possible target molecules include, but are not limited to, molecules functionally related to modulation of integrin function and localization, whose interaction with molecules containing the KAHKKRAD or KARKKHAD or part of it have been shown.

When an aI domain is expressed by a cell, a method of detecting such cell comprises contacting a peptide of the invention with a cell and detecting binding of the peptide to an aI domain on or in the cell. The binding of the peptide to the aI domain may be detected by molecular imaging or any other method known in the art such as those described below. Such binding may be detected by detecting the peptide on or in the cell. Once the cells have been identified, they may be isolated for further characterization and study.

One application of the method is diagnosis of cancer. Generally, the level of binding of the peptide to the aI domain is compared between samples from a test subject and a normal control subject. If the level of the binding of the peptide to the aI domain for the test subject is higher than that for a normal control subject, the test subject is likely to be suffering from cancer or develop cancer.

Another application of the method is to monitor cancer status in a subject. In this method, cancer cells are introduced into a subject using methods commonly employed in the field. The cancer is allowed to progress at the primary site or to metastasis in the subject. A peptide of the invention is then administered to the subject, and the peptide on or in the cancer cells is detected. The location and amount of the bound peptide are indicative of the location and stage of cancer.

An alternative method of monitoring cancer status in a subject involves the steps of administering a peptide of the invention to a subject having cancer cells and detecting the peptide on or in the cancer cells, thereby monitoring the status of the cancer in the subject.

A peptide of the invention can further be used to modulate the biological function or localization of a molecule having an aI domain in vivo and in vitro. The method comprises contacting a peptide of the invention with a molecule having an aI domain, thereby modulating the biological function or localization of the molecule.

Use of the peptides of the invention can be applicable to not only cancer models, but also other pathologies, isolation of specific population of cells, and study of their biology.

In addition, a cell of subline MDA-MB-231-MBF1C-Luc, MDA-MB-231-MBF1C-Luc-GFP, or MDA-MB-231-MM-Luc is within the invention. A “subline” is in here defined as a clonal or non-clonal population of cells derived from a parental cellular population with distinct composition and biological characteristics. These sublines can be obtained according to the methods described in detail below. Because of the unique characteristics demonstrated by these sublines (see below), they are particularly useful for the research of cancer and can be employed in the methods of the invention described herein.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### I. Development of Targeted Non-Invasive Molecular Imaging Modalities and Evaluation of Therapeutic Potential of Integrins in Murine Metastatic Breast Cancer Models

### Summary

Bone and bone marrow are preferential sites for metastasis in multiple forms of cancer. Bidirectional interaction of cancer cells with their microenvironment involves intra- and extra-cellular components in which critical pathways may differ in different patients and cellular populations. Thereby, targeted therapy requires in vivo non-invasive longitudinal profiling of specific molecular components that prevalent cancer subtypes critically rely on.

Metastasis involves multiple steps in which integrin mediated signaling are indispensable. α2β1 is a member of the integrin family of surface receptors present in progenitor populations in breast, prostate, colon and bone marrow. In differentiated cells, α₂β₁ is expressed on platelets, epithelial and mesenchymal cells, among others. The role of α₂β₁ is well described in thrombosis, inflammation, angiogenesis and wound healing. In normal differentiation, α₂β₁ is predominantly localized to terminal ductal epithelia and involved in its branching. In breast cancer, polymorphism in the α₂ gene is associated with progression risk. Importantly, hormonal and growth factor cross talk with this receptor has been reported. Expression, membrane localization and internalization of α₂β₁ are regulated by EGFR that is deregulated in a large percent of breast cancer tumors. α₂β₁ interacting proteases, involved in tissue remodeling and growth factor signaling, are highly expressed in osteoclast, bone, heart and lung. Targeted deletion of α₂ in mice is not lethal and does not result in adverse physiology, allowing the potential to develop tolerated therapeutics against this molecule.

Molecular imaging of xenografts of a panel of luciferase-labeled breast cancer cell lines allows non-invasive in vivo longitudinal study of the biology of these tumors and response to therapeutics based on their molecular signature. Among the cell lines studied in this system are two sublines of the hormone-independent/EGFR (+) MDA-MB-231 breast cancer cells isolated from metastases in femoral bone and musculo-skeletal junction. For each isolate, molecular imaging followed the time course to metastasis in immuno-compromised nude mice after intravenous injection of parental cells. Microscopic examination of in vitro cultures of clonal cells from the isolates revealed changes in morphology as compared to parental cells. Molecule characterization of the integrin profile of the sublines demonstrated greater than 2-4 fold increase in activated α₂β₁ surface expression by flow cytometry that has remained stable after 4 months. Preliminary studies suggest differential binding of these cells to extracellular matrices and anchorage independent of survival and aggregation. In vivo, preliminary longitudinal monitoring of xenografts of these sublines suggest differential tumor growth.

α₂ is among 9 members of the α chain family of integrin whose activation is accompanied by conformational change exposing the aI domain, as well as clustering in specialized microdomains at the cell surface. α₂-specific cyclic peptides have been designed, synthesized and fluorescently labeled, their composition validated by mass spectroscopy, and their increased cell type-specific binding to sublines with increased activated α₂β₁ expression demonstrated by flow cytometry. Effects on biological activity are assessed in in vitro cultures of parental and derived cell lines on multiple extracellular matrices. Preliminary studies are consistent with bioactivity of peptides in terms of adhesion to extracellular matrices.

This model allows development of molecular imaging modalities for detection of α₂β₁ hyperactive populations, characterization of important modulatory signals, as well as evaluation of efficacy of targeted therapeutics in breast cancer subtypes with anomalies of this receptor.

### Results

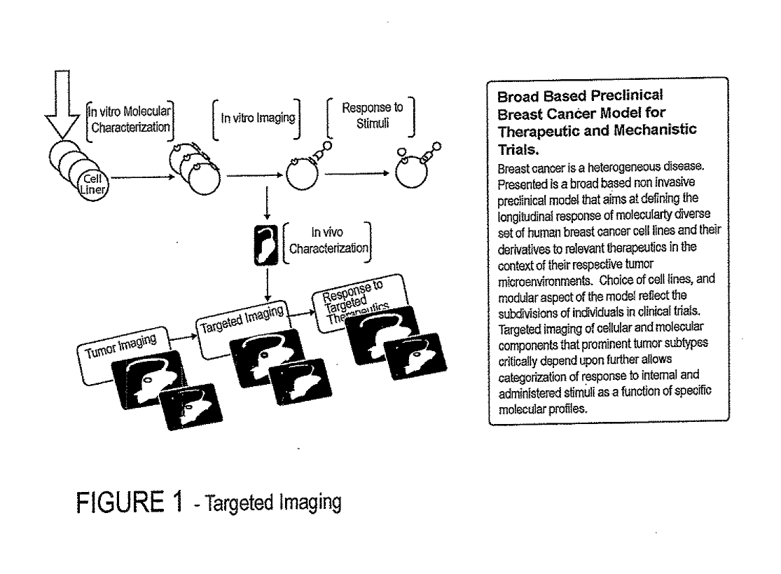

Referring to FIG. 1, breast cancer is a heterogeneous disease. Presented in FIG. 1 is a broad based non-invasive preclinical model that aims at defining the longitudinal response of molecularly diverse set of human breast cancer cell lines and their derivatives to relevant therapeutics in the context of their respective tumor microenvironments. Choice of cell lines and modular aspect of the model reflect the subdivisions of individuals in clinical trials. Targeted imaging of cellular and molecular components that prominent tumor subtypes critically depend upon further allows categorization of response to internal and administered stimuli as a function of specific molecular profiles.

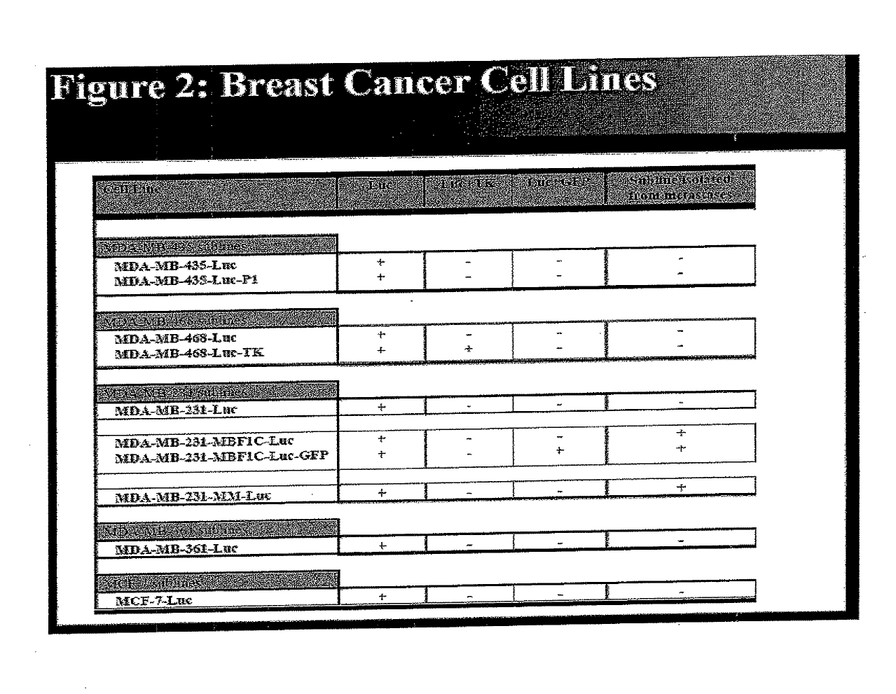

Breast cancer cell lines are shown in FIG. 2.

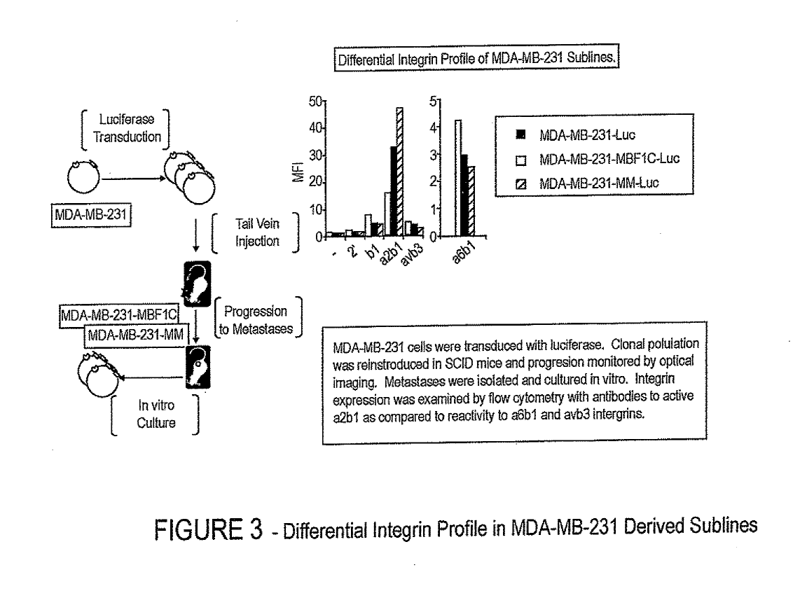

Referring to FIG. 3, MDA-MB-231 cells were transduced with luciferase. Clonal population was reintroduced in SCID mice and progression monitored by optical imaging. Metastases were isolated and culture in vitro. Integrin expression was examined by flow cytometry with antibodies to active α₂β₁, as compared to reactivity to α₆β₁ and αᵥβ₃ integrins.

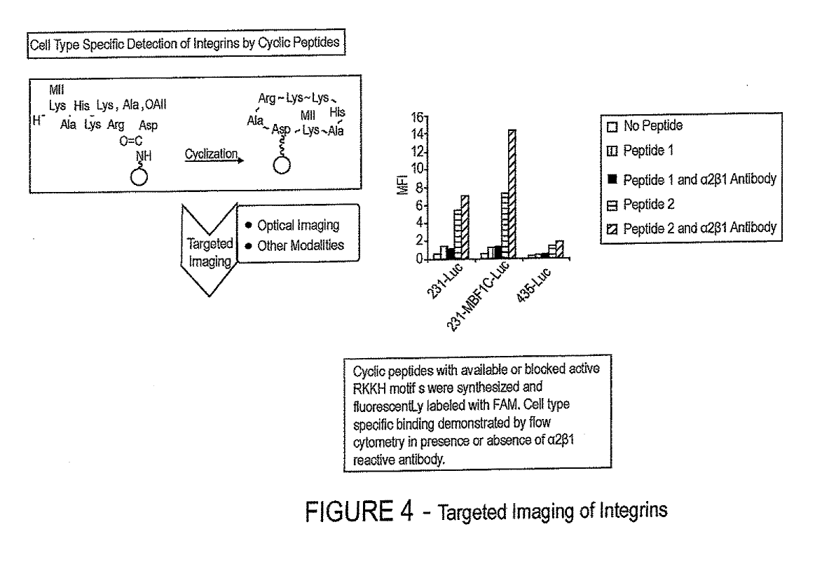

Referring to FIG. 4, cyclic peptides with available or blocked active RKKH motifs were synthesized and fluorescently labeled with FAM. Cell type-specific binding was demonstrated by flow cytometry in presence or absence of α₂β₁ reactive antibody.

### II. Targeted Non-Invasive Molecular Imaging and Evaluation of Therapeutic Potential of Integrins in Murine Metastatic Breast Cancer Models

### Summary of Preliminary Results

Toward development of activity based α₂β₁ imaging and directed therapies, in vitro and in vivo models derived from the MDA-MB-231 breast cancer cell line that can be followed by optical imaging in longitudinal studies have been developed and characterized. Preliminary studies on isolated MDA-MB-231-Luc sublines showed sustained increased α₂β₁ activity, differential binding to extracellular matrices in vitro, and differential tumor growth in vivo.

Conformationally constrained peptides reactive to the aI domain of active α₂β₁ integrin that can be detected by optical imaging in longitudinal studies have also been developed and characterized. Preliminary studies on characterized peptides showed in vitro cell type-specific binding that correlates with α₂β₁ activity, in vitro inhibition of receptor bioactivity in respect to collagen binding, and in vivo tumor-specific uptake.

### Results

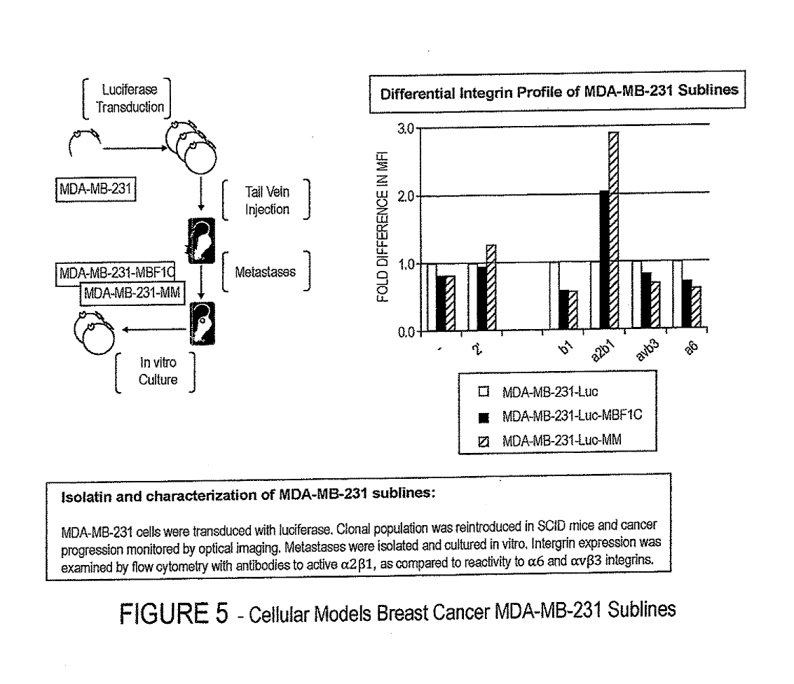

Referring to FIG. 5, MDA-MB-231 sublines were isolated and characterized as follows: MDA-MB-231 cells were transduced with luciferase. Clonal population was reintroduced in SCID mice and cancer progression monitored by optical imaging. Metastases were isolated and cultured in vitro. Integrin expression was examined by flow cytometry with antibodies to active α₂β₁, as compared to reactivity to α₆ and αᵥβ₃ integrins.

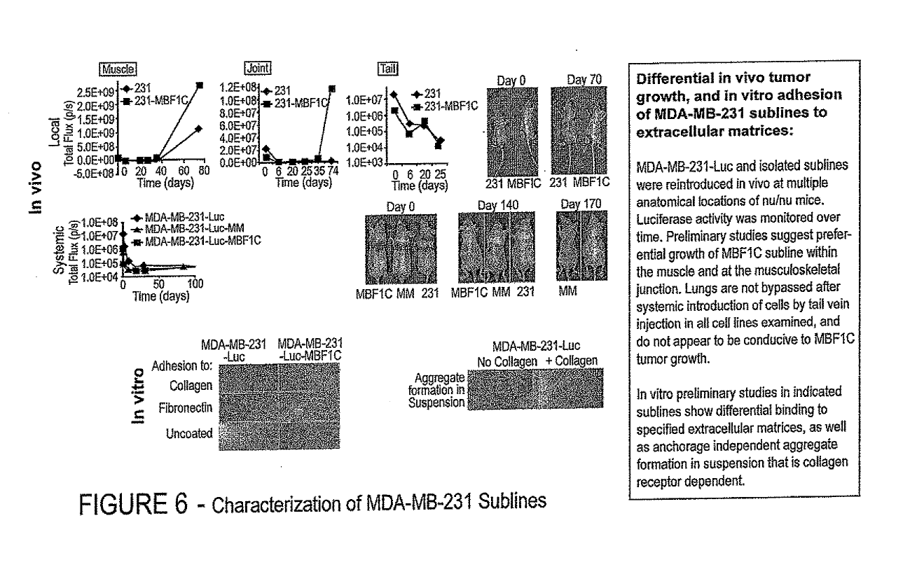

Referring to FIG. 6, differential in vivo tumor growth, and in vitro adhesion of MDA-MB-231 sublines to extracellular matrices were demonstrated. MDA-MB-231-Luc and isolated sublines were reintroduced in vivo at multiple anatomical locations of nu/nu mice. Luciferase activity was monitored over time. Preliminary studies suggest preferential growth of MBF1C sublime within the muscle and at the musculoskeletal junction. Lungs were not bypassed after systemic introduction of cells by tail vein injection in all cell lines examined, and did not appear to be conducive to MBF1C tumor growth.

In vitro preliminary studies in indicated sublines show differential binding to specified extracellular matrices, as well as anchorage-independent aggregate formation in suspension that is collagen receptor-dependent.

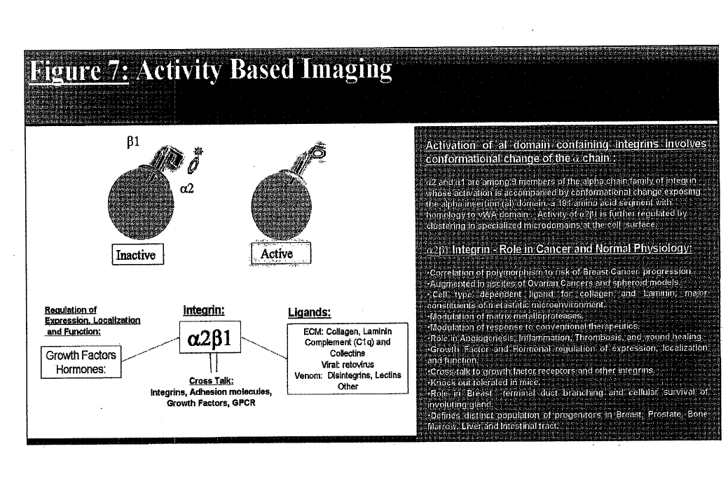

Referring to FIG. 7, activation of aI domain containing integrins involves conformational change of the aI chain. α₂ and α₁ are among 9 members of the α chain family of integrin whose activation is accompanied by conformational change exposing the alpha insertion (aI) domain, a 191 amino acid segment with homology to vWA domain. Activity of α₂β₁ is further regulated by clustering in specialized microdomains at the cell surface.

α₂β₁ integrin play important roles in cancer and normal physiology, including correlation of polymorphism to risk of breast cancer progression; augment in ascites of ovarian cancers and spheroid models; cell type-dependent ligand for collagen and Laminin; major constituents of metastatic microenvironment; modulation of matrix metalloproteases; modulation of response to conventional therapeutics; angiogenesis, inflammation, thrombosis, and wound healing; growth factor and hormonal regulation of expression, localization and function; cross-talk to growth factor receptors and other integrins; knock-out tolerance in mice; breast terminal duct branching and cellular survival of involuting gland; defining distinct population of progenitors in breast, prostate, bone marrow, liver and intestinal tract.

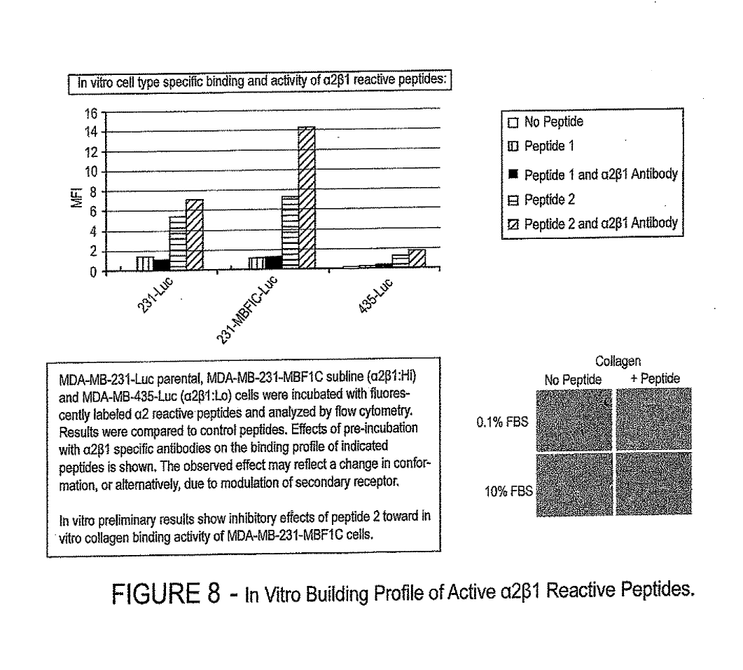

Referring to FIG. 8, MDA-MB-231-Luc parental, MDA-MB-231-MBF1C subline (α₂β₁:Hi) and MDA-MB-435-Luc (α₂β₁:Lo) cells were incubated with fluorescently labeled α₂ reactive peptides and analyzed by flow cytometry. Results were compared to control peptides. Effects of pre-incubation with α₂β₁-specific antibodies on the binding profile of indicated peptides are shown. The observed effect may reflect a change in conformation, or alternatively, due to modulation of secondary receptor.

In vitro preliminary results show inhibitory effects of Peptide 2 (see below) toward in vitro collagen binding activity of MDA-MB-231-MBF1C cells.

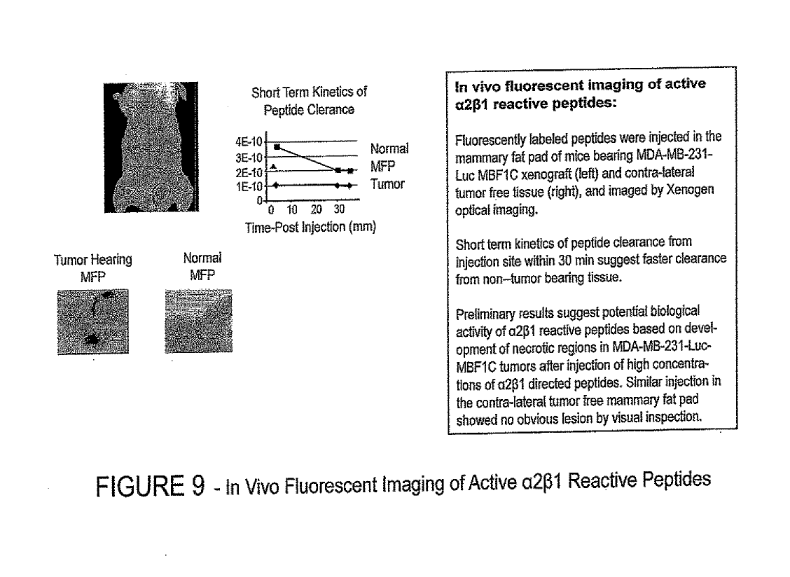

Referring to FIG. 9, fluorescently labeled peptides were injected in the mammary fat pad of mice bearing MDA-MB-231-Luc-MBF1C xenograft (left) and contra-lateral tumor-free tissue (right), and imaged by Xenogen optical imaging. Short term kinetics of peptide clearance from injection site within 30 min suggest faster clearance from non-tumor-bearing tissue. Preliminary results suggest potential biological activity of α₂β₁-reactive peptides based on development of necrotic regions in MDA-MB-231-Luc-MBF1C tumors after injection of high concentrations of α₂β₁-directed peptides. Similar injection in the contra-lateral tumor-free mammary fat pad showed no obvious lesion by visual inspection.

### III

### A. Functional Imaging and Therapeutics Targeted to Active Integrins:

α₂ and α₁ Activation is Accompanied by Conformational Change:

Exposing the aI domain, thereby allowing design of high avidity, activity-specific ligands.

Significance:
1. Functional imaging of active integrins: Early detection of population of cancer cells and tumors with active integrins.
2. Therapeutics targeted to active integrins: Targeting of cellular populations dependent on functional activity of integrins.
3. Basic biology: Definition of cellular population dependent on active integrin function; definition of integrin dependent survival mechanisms in above populations.

### B. Functional Importance of α₂β₁ in Normal Physiology

Role of α₂β₁ in Progenitor Cellular Populations and Respective Biology:
α₂β₁ defines distinct population of progenitors in breast, prostate, colon, liver and bone marrow.
Expression of α₂β₁ in in vitro and in vivo models of ovarian cancer is not uniform and may prove important toward characterization and function of distinct subset of progenitor cells in ovarian cancers.

Intracellular Signal Transduction and Molecular Regulators:
Pathways mediating transduction of signal downstream and upstream of α₂β₁, such as MAPK and PI3K, play important functions in biology of progenitor cells and cellular survival.
α₂β₁ is hormonally regulated.

α₂β₁ Cross Talks:
α₂β₁ modulates other integrins such as αᵥβ₃ and α₁β₁.
α₂β₁ is functionally targeted to membrane microdomains.
Expression, membrane localization and internalization of α₂β₁ are regulated by EGF and ErbB.
Cross talk with growth, survival and differentiation factor such as TGFβ and VEGF.

Genetic Models:
Knock out of α₂ is tolerated in mice.

### C. Functional Importance of α₂β₁ in Ovarian Cancers

Differential α₂β₁ Expression and Function:
Polymorphism in the α₂ gene is associated with cancer progression risk.
Primary tumors, associated endothelial lining, and ovarian cancer cell lines differentially utilize α₂β₁ integrin as compared to normal tissue.
Level of α₂β₁ is augmented in patient's ascites in advanced stages.
In vitro spheroids models, expression of α₂β₁ remains elevated in human ovarian cancer cell lines, as opposed to primary non-malignant cells.

α₂β₁ Ligands and Metastasis:
Major collagen and laminin receptor.
Critical components of mesothelial targets for ovarian cancer metastases.
Increased α₂β₁ expression correlates and its inhibition with blocking antibody modulates expression and activation MMP2 and MMP9.
Role of α₂β₁ in processes important to cancer progression such as angiogenesis, inflammation, thrombosis, and wound healing.

Response to Conventional Chemotherapeutics:
Response to taxanes and radiation is altered in spheroids cultures.
Integrin/aspase-independent cell death may be important.
   D. aI targeted Peptides
Venom of Bethrops jararaca inhibits interaction of α₂β₁ to collagen due to the action of jararhagin.
Ligand binding domain of jararhagin is distinct from that of RGD containing disintegrins.
Inhibition of collagen binding is mediated by a distinct domain with specificity to the α chain aI domain.
The central RKKH motif is required for aI domain specificity.
Binding is dependent on the integrin MIDAS domain and presence of Mg²⁺.
Targeting of the metalloprotease to the aI domain allows proteolytic action on associated molecules.
Binding induces conformational changes in the α chain.
RKKH motif is present in other molecules including FN-C/H II peptide where its substitution results in inhibition of tumor growth, as well as in PDGF-B loopIII and other intracellular proteins.

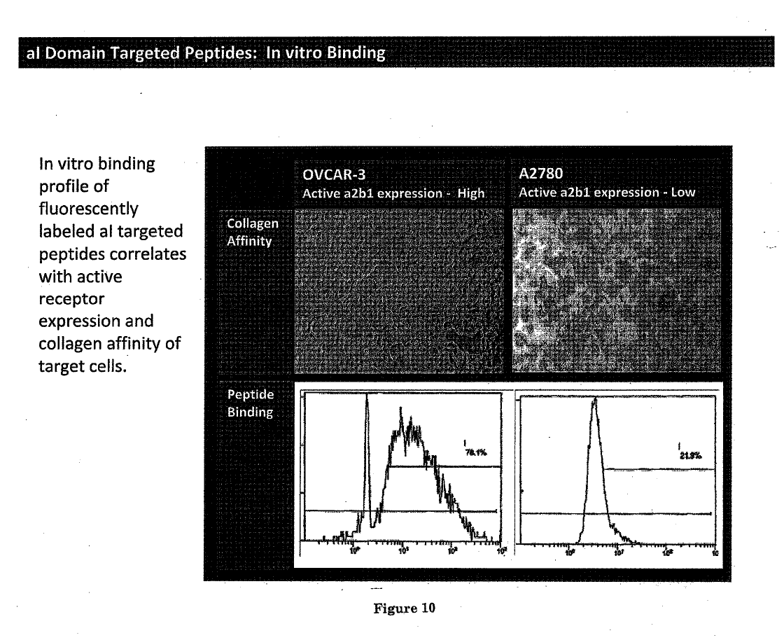

Referring to FIG. 10, in vitro binding profile of fluorescently labeled aI targeted peptides correlates with active receptor expression and collagen affinity of target cells.

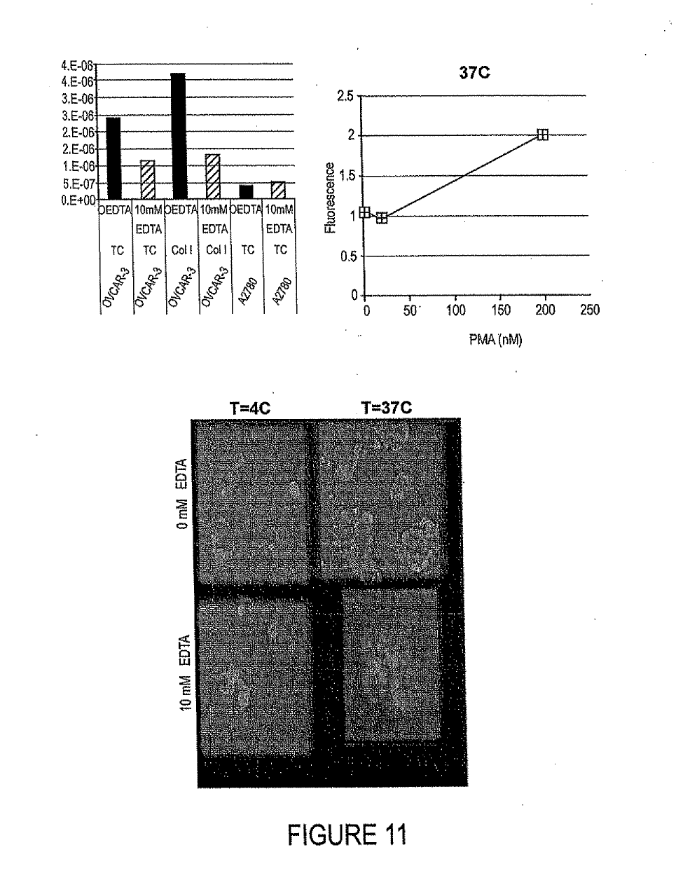

Referring to FIG. 11, in vitro binding of aI targeted peptides were inhibitable by cation chelators such as EDTA. Confocal imaging aI targeted peptides showed active receptor patches at the cell surface that are internalized in localized compartments at 37° C., that is inhibitable by EDTA and decreased temperature. Conversely, activation of the receptor by PMA increased the level of cell-associated peptide as shown for OVCAR-3 cells.

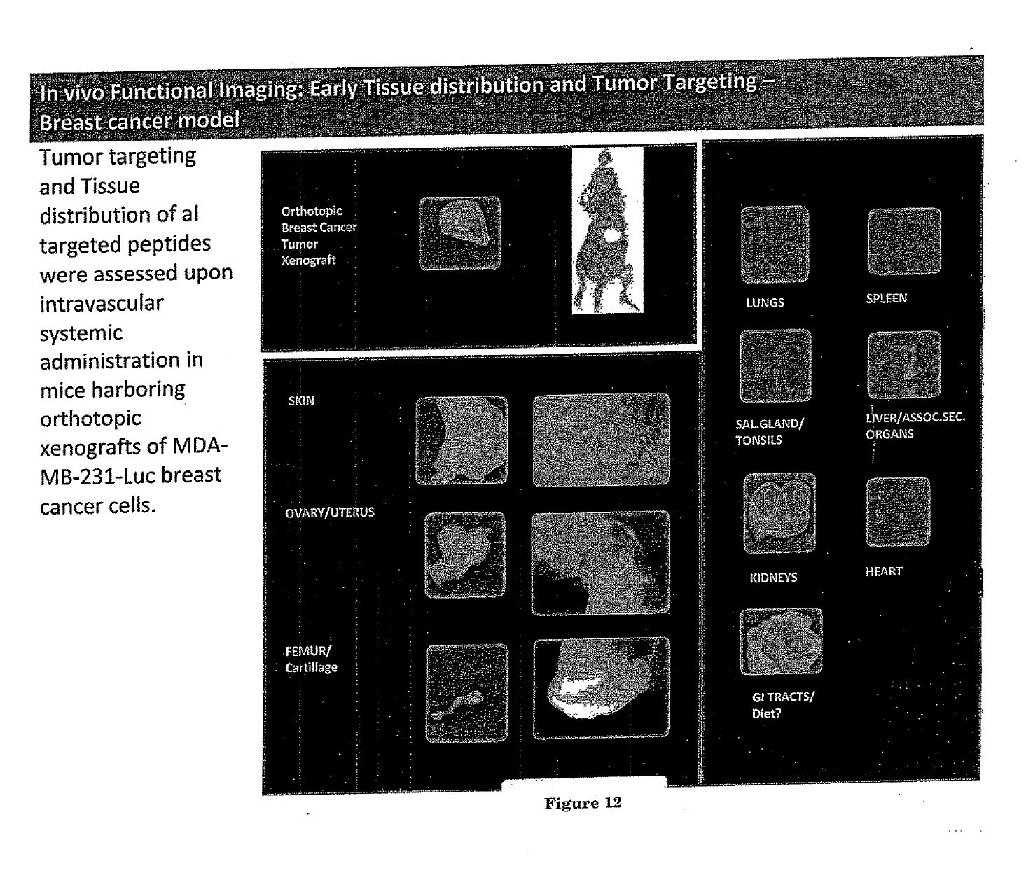

Referring to FIG. 12, tumor targeting and tissue distribution of aI targeted peptides were assessed upon intravascular systemic administration in mice harboring orthotopic xenografts of MDAMB-231-Luc breast cancer cells.

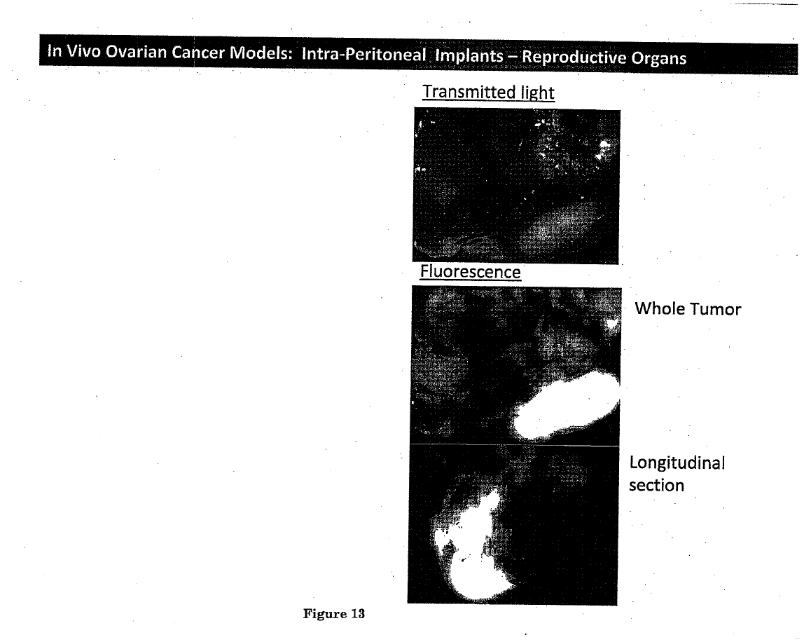

Referring to FIG. 13, a tumor arose after transplant of A2780 ovarian cancer cells in the peritoneum of young female Nu/Nu mice. Implant developed into solid tumor in addition to bloody ascites. Solid tumor was localized around the ovary and uterus. Intraperitoneal injection of aI targeted peptide resulted in its differential targeting to the solid tumor as compared to other null organs. Fluorescent images showed non-uniform distribution of peptide 1 on the dissected tumor as visualized by stereoscopic fluorescent microscopy.

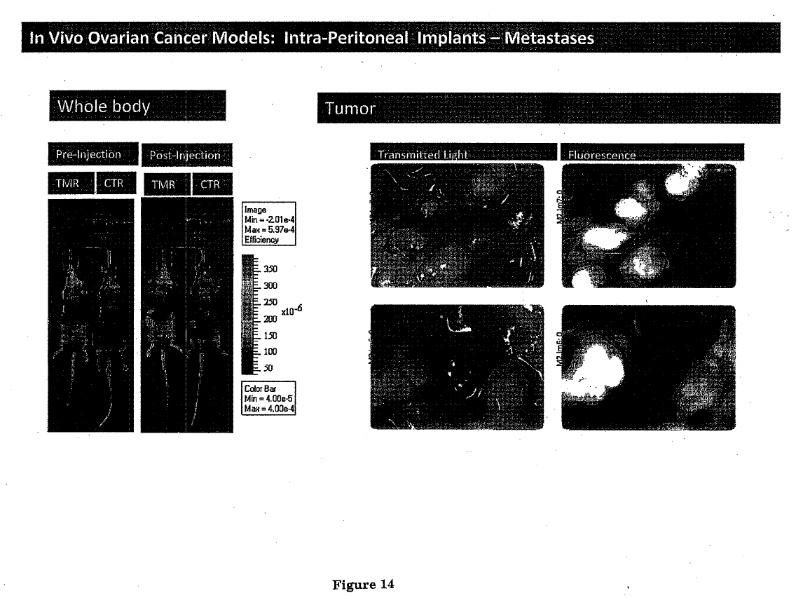

Referring to F FIG. 14, intra-peritoneally implanted xenografts of human ovarian cancer cells (A2780) resulted in metastatic-like nodules around intestinal tracts. Peptide 1 (see below) was administered intra-peritoneally, and whole body fluorescent imaging was performed in living animals. Fluorescent stereoscope photographs of above nodules in dissected animals at one hour post peptide administration are shown. Caption indicates exposure times. Fluorescent images were colored post acquisition.

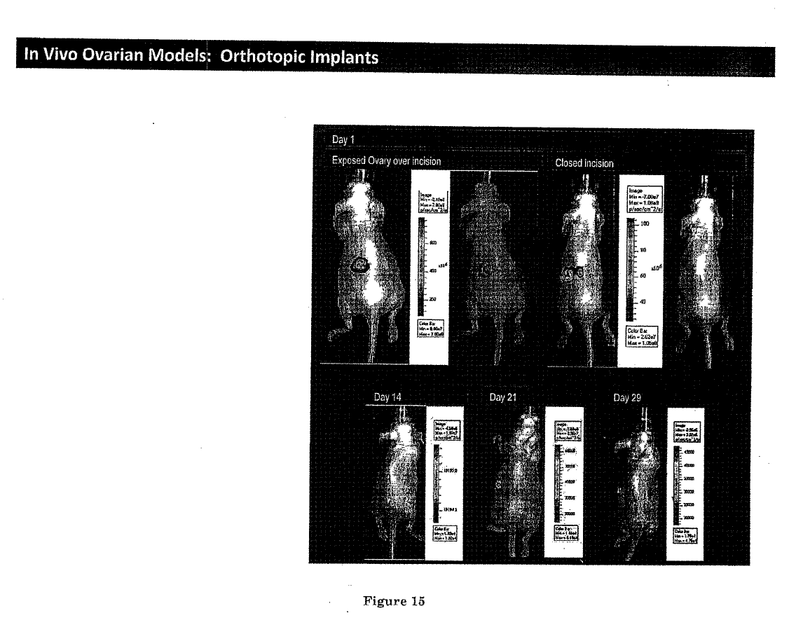

Referring to FIG. 15, the ability to implant human cancer cells locally at the ovary of middle-aged Nu/Nu mice is shown. Luciferase transduced cells (MDAMB-231-Luc breast cancer) were mixed with luciferin and locally injected in the left ovary. Lower panel shows viability and lack of morbidity in mice recovering from survival surgery. Persistence and viability of cells was shown up to 29 days. Cancer progression and activity state of integrins can be monitored in implants of human ovarian cancer cells.

### IV

### A. Peptides Developed and Used in Described Studies Include:

Peptide 1: FAM-KAHKKRAD Cyclic: Sidechain Backbone

Peptide 2: FAM-KAHKKRAD Cyclic: Backbone Backbone

Peptide 3: FAM-KARKKHAD Cyclic: Backbone Backbone

Peptide 4: Biotin-KAHKKRAD Cyclic: Sidechain Backbone

Peptide 5: Biotin-KAHKKRAD Cyclic: Backbone Backbone

Peptide 6: Biotin-KARKKHAD Cyclic: Backbone Backbone

Peptide 1: FAM-KAHKKRAD Linear

Peptide 2: FAM-KAHKKRAD Linear

Peptide 3: FAM-KARKKHAD Linear

Peptide 4: Biotin-KAHKKRAD Linear

Peptide 5: Biotin-KAHKKRAD Linear

Peptide 6: Biotin-KARKKHAD Linear

Core recognition sequences of the peptides are based on studies of jararhagin metalloproteinase disintegrin, as described above. Orientation and additional sequences surrounding the recognition motifs allow proper cyclization and potentially increase the ligand spectrum to other molecules important to integrin function and trafficking, such as RapGAPs. Constraining peptides by cyclization allows increased stability and proper three dimensional conformation. Multiple cyclization methods allow study and definition of optimal ligand binding structure. FAM fluorescent tag has been added for detection of the molecule in preliminary in vitro and in vivo studies, and can be replaced to other moieties amenable to clinical imaging modalities. Biotin moiety is added to identify the spectrum of ligands and linkage to other molecules.

### B. Cellular and Animal Models:

Peptides used in the studies have been applied to two models of hormone dependent cancers, namely ovarian and breast cancers. In the breast cancer, a sublime of MDA-MB-231-Luc, obtained from metastasis of the parental xenograft, has been characterized and shown to express increased α₂β₁ activity as compared to its parental line. Xenografts of above and other available luciferase transduced breast cancer cells have been implanted in vivo orthotopically at the mammary fat pad, or other anatomical locations.

In the ovarian cancer model, two specific cell lines (A2780 and OVCAR-3) have been used. For in vivo studies, cells have been introduced directly to the peritoneal cavity by ip injection, or orthotpically implanted surgically in the ovary.

### C. Response to Cellular Stress Stimuli:

1) Glucose deprivation
2) Inhibition of mTOR

This study aims at definition of biological mechanisms responsible for potential therapeutic potential of aI domain targeted peptides. Cellular survival mechanisms are prerequisite to differentiation, growth and proliferation. The questions are whether integrin activity modulates caspase-independent cellular survival in cells important to cancer initiation and progression, and whether aI targeted peptides interaction, localization and function modulate the cellular survival of critical cells important to cancer initiation and progression. In addition to their roles in normal physiology, cross regulation of apoptotic and autophagic cell death and survival pathways have been shown, and have proved important in cancer initiation, progression and resistance to chemo- and immune-therapeutics. Cellular signal transduction pathways regulating these processes are in part regulated by integrin function. Furthermore, aI targeted peptides presented here have homology to molecules that regulate integrin function, vesicular targeting and cellular survival.

### D. In Vivo Tumor Targeting, Distribution and Clearance Kinetics of Developed Peptides:

FIG. 12 shows tumor targeting of Peptide 1 and its tissue distribution in female Nu/Nu mice harboring MDA-MB-231-Luc with an orthotopic tumor in the left mammary fat pad. Early distribution of peptide to other tissue utilizing the active receptor is shown. Indicated tissues and organs were isolated post sacrifice of the animal and peptide visualized by stereoscopic fluorescent microscopy or xenogeny biofluorescence scanning.

### REFERENCES

1. Sawyers C. Targeted cancer therapy. Nature. 2004 Nov. 18; 432(7015):294-7. Review.
2. Pegram M D, Konecny G, Slamon DJ. The molecular and cellular biology of HER2/neu gene amplification/overexpression and the clinical development of herceptin (trastuzumab) therapy for breast cancer. Cancer Treat Res. 2000; 103:57-75. Review.
3. Moasser M M. Targeting the function of the HER2 oncogene in human cancer therapeutics. Oncogene. 2007 Oct. 11; 26(46):6577-92. Review.
4, Dalgin G S, Alexe G, Scanfeld D, Tamayo P, Mesirov J P, Ganesan S, DeLisi C, Bhanot G. Portraits of breast cancer progression. BMC Bioinformatics. 2007 Aug. 6; 8:291.
5. Zhou Y, Yau C, Gray J W, Chew K, Dairkee S H, Moore D H, Eppenberger U, Eppenberger-Castori S, Benz CC. Enh awed NF kappa B and AP-1 transcriptional activity associated with antiestrogen resistant breast cancer. BMC Cancer. 2007 Apr. 3; 7:59.
6. Langsenlehner U, Renner W, Yazdani-Biuki B, Eder T, Wascher T C, Paulweber B, Clar H, Hofmann G, Samonigg H, Krippl P. Integrin alpha-2 and beta-3 gene polymorphisms and breast cancer risk. Breast Cancer Res Treat. 2006 May; 97(1):67-72.
7. Ayala F, Corral J, González-Conejero R, Sánchez I, Moraleda J M, Vicente V. Genetic polymorphisms of platelet adhesive molecules; association with breast cancer risk and clinical presentation. Breast Cancer Res Treat. 2003 July; 80(2):145-54.
8. Di Paola J, Jugessur A, Goldman T, Reiland J, Tallman D, Sayago C, Murray J C. Platelet glycoprotein I(b)alpha and integrin alpha2 beta1 polymorphisms: gene frequencies and linkage disequilibrium in a population diversity panel. J Thromb Haemost. 2005 July; 3(7):1511-21.
9. Mercurio A M. Lessons from the alpha2 integrin knockout mouse, Am J. Pathol. 2002 July; 161(1):3-6. Review.
10. Chen J, Diacovo T G, Grenache D G, Santoro S A, Zutter M M. The alpha(2) integrin subunit-deficient mouse: a multifaceted phenotype including defects of branching morphogenesis and hemostasis. Am J Pathol. 2002 July; 161(1):337-44.
11. Grenache D G, Zhang Z, Wells L E, Santoro S A, Davidson J M, Zutter M M. Wound healing in the alpha2beta1 integrin-deficient mouse: altered keratinocyte biology and dysregulated matrix metalloproteinase expression. J Invest Dermatol. 2007 February; 127(2):455-66.
12. Emsley J, King S L, Bergelson J M, Liddington RC. Crystal structure of the I domain from integrin alpha2beta1. J Biol Chem. 1997 Nov. 7; 272(45):28512-7.
13. Sawhney R S, Cookson M M, Omar Y, Hauser J, Brattain M G. Integrin alpha2-mediated ERK and calpain activation play a critical role in cell adhesion and motility via focal adhesion kinase signaling: identification of a novel signaling pathway. J Biol Chem. 2006 Mar. 31; 281(13):8497-510.
14. Vuoristo M, Vihinen P, Vlaykova T, Nylund C, Heino J, Pyrhönen S. Increased gene expression levels of collagen receptor integrins are associated with decreased survival parameters in patients with advanced melanoma. Melanoma Res. 2007 August; 17(4):215-23.
15. Baciu P C, Suleiman E A, Deryugina E I, Strongin A Y. Membrane type-1 matrix metalloproteinase (MT1-MMP) processing of pro-alphav integrin regulates cross-talk between alphavbeta3 and alpha2beta1 integrins in breast carcinoma cells. Exp Cell Res. 2003 Nov. 15; 291(1):167-75.
16. Leitinger B, Hohenester E. Mammalian collagen receptors. Matrix Biol. 2007 April; 26(3):146-55. Review.
17. Givant-Horwitz V, Davidson B, Reich R. Laminin-induced signaling in tumor cells. Cancer Lett. 2005 Jun. 1; 223(1):1-10. Review.
18. Xiao G, Wang D, Benson M D, Karsenty G, Franceschi R T. Role of the alpha2-integrin in osteoblast-specific gene expression and activation of the Osf2 transcription factor. J Biol Chem. 1998 Dec. 4; 273(49):32988-94.
19. Vihinen P, Riikonen T, Laine A, Heino J. Integrin alpha 2 beta 1 in tumorigenic human osteosarcoma cell lines regulates cell adhesion, migration, and invasion by interaction with type I collagen. Cell Growth Differ. 1996 April; 7(4):439-47.
20. Ning Y, Buranda T, Hudson LG. Activated epidermal growth factor receptor induces integrin alpha2 internalization via caveolae/raft-dependent endocytic pathway. J Biol Chem. 2007 Mar. 2; 282(9):6380-7.
21. Ning Y, Zeineldin R, Liu Y, Rosenberg M, Stack M S, Hudson LG. Down-regulation of integrin alpha2 surface expression by mutant epidermal growth factor receptor (EGFRvIII) induces aberrant cell spreading and focal adhesion formation. Cancer Res. 2005 Oct. 15; 65(20):9280-6.
22. Lindberg L E, Hedjazifar 5, Baeckström D. c-erbB2-induced disruption of matrix adhesion and morphogenesis reveals a novel role for protein kinase B as a negative regulator of alpha(2)beta(1) integrin function. Mol Biol Cell. 2002 August; 13(8):2894-908.
23. Zhang Z, Ramirez N E, Yankeelov T E, Li Z, Ford L E, Qi Y, Pozzi A, Zutter M M. {alpha}2{beta}1 integrin expression in the tumor microenvironment enhances tumor angiogenesis in a tumor-cell specific manner. Blood. 2008 Feb. 15; 111(4):1980-8.
24. Hong Y K, Lange-Asschenfeldt B, Velasco P, Hirakawa S, Kunstfeld R, Brown L F, Bohlen P, Senger D R, Detmar M. VEGF-A promotes tissue repair-associated lymphatic vessel formation via VEGFR-2 and the alpha1beta1 and alpha2beta1 integrins. FASEB J. 2004 July; 18(10):1111-3.
25. Klekotka P A, Santoro S A, Wang H, Zutter M M. Specific residues within the alpha 2 integrin subunit cytoplasmic domain regulate migration and cell cycle progression via distinct MAPK pathways. J Biol Chem. 2001 Aug. 24; 276(34):32353-61.
26. Klekotka P A, Santoro S A, Zutter M M. alpha 2 integrin subunit cytoplasmic domain-dependent cellular migration requires p38 MAPK. J Biol Chem. 2001 Mar. 23; 276(12):9503-11.
27. Sawhney R S, Cookson M M, Sharma B, Hauser J, Brattain M G. Autocrine transforming growth factor alpha regulates cell adhesion by multiple signaling via specific phosphorylation sites of p70S6 kinase in colon cancer cells. J Biol Chem. 2004 Nov. 5; 279(45):4737.9-90.
28. Jin S, White E. Role of autophagy in cancer: management of metabolic stress. Autophagy. 2007 January-February; 3(1):28-31. Review.
29. Motyl T, Gajkowska B, Zarzyńska J, Gajewska M, Lamparska-Przybysz M. Apoptosis and autophagy in mammary gland remodeling and breast cancer chemotherapy. J Physiol Pharmacol. 2006 November; 57 Suppl 7:17-32. Review.
30. Karantza-Wadsworth V, White E. Role of autophagy in breast cancer. Autophagy. 2007 November-December; 3(6):610-3.
31. Jin S. p53, Autophagy and tumor suppression. Autophagy. 2005 October-December; 1(3):171-3. Review.
32. Abedin M J, Wang D, McDonnell M A, Lehmann U, Kelekar A. Autophagy delays apoptotic death in breast cancer cells following DNA damage. Cell Death Differ. 2007 March; 14(3):500-10.
33. Tan E Y, Campo L, Han C, Turley H, Pezzella F, Gatter K C, Harris A L, Fox S B. BNIP3 as a progression marker in primary human breast cancer; opposing functions in in situ versus invasive cancer. Clin Cancer Res. 2007 Jan. 15; 13(2 Pt 1):467-74.
34. Qadir M A, Kwok B, Dragowska W H, To K H, Le D, Bally M B, Gorski S M. Macroautophagy inhibition sensitizes tamoxifen-resistant breast cancer cells and enhances mitochondrial depolarization. Breast Cancer Res Treat. 2008 Jan. 3; [Epub ahead of print].
35. Kim K W, Mutter R W, Cao C, Albert J M, Freeman M, Hallahan D E, Lu B. Autophagy for cancer therapy through inhibition of pro-apoptotic proteins and mammalian target of rapamycin signaling. J Biol Chem. 2006 Dec. 1; 281(48):36883-90.
36. Arthur C R, Gupton J T, Kellogg G E, Yeudall W A, Cabot M C, Newsham I F, Gewirtz D A. Autophagic cell death, polyploidy and senescence induced in breast tumor cells by the substituted pyrrole JG-03-14, a novel microtubule poison. Biochem Pharmacol. 2007 Oct. 1; 74(7):981-91.
37. Qu X, Yu J, Bhagat G, Furuya N, Hibshoosh H, Troxel A, Rosen J, Eskelinen E L, Mizushima N, Ohsumi Y, Cattoretti G, Levine B. Promotion of tumorigenesis by heterozygous disruption of the beclin 1 autophagy gene. J Clin Invest. 2003 December; 112(12):1809-20.
38. Liang X H, Jackson S, Seaman M, Brown K, Kempkes B, Hibshoosh H, Levine B. Induction of autophagy and inhibition of tumorigenesis by beclin 1. Nature. 1999 Dec. 9; 402(6764672-6.
39. Karantza-Wadsworth V, Patel S, Kravchuk O, Chen G, Mathew R, Jin S, White E. Autophagy mitigates metabolic stress and genome damage in mammary tumorigenesis. Genes Dev. 2007 Jul. 1; 21(13):1621-35.
40. Mathew R, Kongara S, Beaudoin B, Karp C M, Bray K, Degenhardt K, Chen G, Jin S, White E. Autophagy suppresses tumor progression by limiting chromosomal instability. Genes Dev. 2007 Jun. 1; 21(11):1367-81.
41. Edick M J, Tesfay L, Lamb L E, Knudsen B S, Miranti C K. Inhibition of integrin-mediated crosstalk with epidermal growth factor receptor/Erk or Src signaling pathways in autophagic prostate epithelial cells induces caspase-independent death. Mol Biol Cell. 2007 July; 18(7):2481-90.
42. Chen G C, Lee J Y, Tang H W, Debnath J, Thomas S M, Settleman J. Genetic interactions between Drosophila melanogaster Atg1 and paxillin reveal a role for paxillin in autophagosome formation. Autophagy. 2008 January-February; 4(1):37-45.
43. Jezierska A, Matysiak W, Motyl T. ALCAM/CD166 protects breast cancer cells against apoptosis and autophagy. Med Sci Monit. 2006 August; 12(8):BR263-73.
44. Schliess F, Reissmann R, Reinehr R, vom Dahl S, Häussinger D. Involvement of integrins and Src in insulin signaling toward autophagic proteolysis in rat liver. J Biol Chem. 2004 May 14; 279(20):21294-301.
45. Wullschleger S, Loewith R, Oppliger W, Hall M N. Molecular organization of target of rapamycin complex 2. J Biol Chem. 2005 Sep. 2; 280(35):30697-704.
46. Reggiori F, Monastyrska I, Shintani T, Klionsky D J. The actin cytoskeleton is required for selective types of autophagy, but not nonspecific autophagy, in the yeast Saccharomyces cerevisiae. Mol Bial Cell. 2005 December; 16(12):5843-56.
47. Bursch W, Hochegger K, Torok L, Marian B, Ellinger A, Hermann R S. Autophagic and apoptotic types of programmed cell death exhibit different fates of cytoskeletal filaments, J Cell Sci. 2000 April; 113 (Pt 7):1189-98.
48. Aplin A, Jasionowski T, Tuttle. DL, Lenk S E, Dunn W A Jr. Cytoskeletal elements are required for the formation and maturation of autophagic vacuoles. J Cell Physiol. 1992 September; 152(3):458-66.
49. Bray P F. Integrin polymorphisms as risk factors for thrombosis. Thromb Haemost. 1999 August; 82(2):337-44. Review.
50. Chen J, Diacovo T G, Grenache D G, Santoro S A, Zutter M M. The alpha(2) integrin subunit-deficient mouse: a multifaceted phenotype including defects of branching morphogenesis and hemostasis. Am J. Pathol. 2002 July; 161(1):337-44.
51. Corbi A L, Jensen U B, Watt F M. The alpha2 and alpha5 integrin genes: identification of transcription factors that regulate promoter activity in epidermal keratinocytes. FEES Lett. 2000 Jun. 2; 474(2-3):201-7.
52. Ramakrishnan S, Nguyen T M, Subramanian I V, Kelekar A. Autophagy and angiogenesis inhibition. Autophagy. 2007 September-October; 3(5):512-5.
53. Chau Y P, Lin S Y, Chen J H, Tai M H. Endostatin induces autophagic cell death in EAhy926 human endothelial cells. Histol Histopathol. 2003 July; 18(3):715-26.
54. Nagakawa O, Akashi T, Hayakawa Y, Junicho A, Koizumi K, Fujiuchi Y, Furuya Y, Matsuda T, Fuse H, Saiki I. Differential expression of integrin subunits in DU-145/AR prostate cancer cells. Oncol Rep. 2004 October; 12(4):837-41.
55. Type T, Jayasree K, Sudhakaran P R. Modulation of alpha2beta1 integrin changes during mammary gland development by beta-oestradiol. Biochim Biophys Acta. 2001 Jan. 15; 1499(3):232-41.
56. Ryu J S, Majeska R J, Ma Y, LaChapelle L, Guller S. Steroid regulation of human placental integrins: suppression of alpha2 integrin expression in cytotrophoblasts by glucocorticoids. Endocrinology. 1999 September; 140(9):3904-8.
57. Novaro V, Roskelley C D, Bissell M J. Collagen-IV and laminin-1 regulate estrogen receptor alpha expression and function in mouse mammary epithelial cells. J Cell Sci. 2003 Jul. 15; 116(Pt 14):2975-86.
58. Förster C, Mäkela S, Wärri A, Kietz S, Becker D, Hultenby K, Warner M, Gustafsson J A. Involvement of estrogen receptor beta in terminal differentiation of mammary gland epithelium. Proc Natl Acad Sci USA. 2002 Nov. 26; 99(24):15578-83.
59. Iype T, Jayasree K, Sudhakaran P R. Modulation of alpha2beta1 integrin changes during mammary gland development by beta-oestradiol. Biochim Biophys Acta. 2001 Jan. 15; 1499(3):232-41.
60. Zutter M M, Santoro S A, Painter A S, Tsung Y L, Gafford A. The human alpha 2 integrin gene promoter. Identification of positive and negative regulatory elements important for cell-type and developmentally restricted gene expression. J Biol Chem. 1994 Jan. 7; 269(1):463-9.
61. Radisky D C, Bissell M J. NF-kappaB links oestrogen receptor signalling and EMT. Nat Cell Biol. 2007 April; 9(4):361-3.
62. Shackleton M, Vaillant F, Simpson K J, Stingl J, Smyth G K, Asselin-Labat M L, WuL, Lindeman G J, Visvader J E. Generation of a functional mammary gland from a single stem cell. Nature. 2006 Jan. 5; 439(7072):84-8.
63. Vaillant F, Asselin-Labat M L, Shackleton M, Lindeman G J, Visvader J E. The emerging picture of the mouse mammary stem cell. Stem Cell Rev, 2007 June; 3(2):114-23. Review.
64. Wagers A J, Weissman I L. Differential expression of alpha2 integrin separates long-term and short-term reconstituting Lin-/loThy1.1(lo)c-kit+Sca-1+ hematopoietic stem cells. Stem Cells. 2006 April; 24(4):1087-94.
65. Xin L, Lukacs R U, Lawson D A, Cheng D, Witte O N. Self-renewal and multilineage differentiation in vitro from murine prostate stem cells. Stem Cells. 2007 November; 25(11):2760-9.
66. Mey A, Gandrillon O, McNagny K M, Clegg D O, Samarut J. The v-erbA oncogene blocks expression of alpha2/beta1 integrin a normal inhibitor of erythroid progenitor proliferation. Oncogene. 2002 Apr. 25; 21(18):2864-72.
67. Koenig 5, Krause P, Drabent B, Schaeffner I, Christ B, Schwartz P, Unthan-Fechner K, Probst I. The expression of mesenchymal, neural and haematopoietic stem cell markers inadult hepatocytes proliferating in vitro. J Hepatol. 2006 June; 44(6):1115-24.
68. Chen C P, Lee M Y, Huang J P, Aplin J D, Wu Y H, Hu C S, Chen P C, Li H, Hwang S M, Liu S H, Yang Y C. Trafficking of Multipotent Mesenchymal Stromal Cells From Maternal Circulation Through the Placenta Involves VEGFR-1 and Integrins. Stem Cells. 2008 February; 26(2):550-61.
69. Mueller F J, Serobyan N, Schraufstatter I U, DiScipio R, Wakeman D, Loring J F, Snyder E Y, Khaldoyanidi S K. Adhesive interactions between human neural stem cells and inflamed human vascular endothelium are mediated by integrins. Stem Cells. 2006 November; 24(11):2367-72.
70. Heckmann L, Fiedler J, Mattes T, Brenner R E. Mesenchymal progenitor cells communicate via alpha and beta integrins with a three-dimensional collagen type I matrix. Cells Tissues Organs. 2006; 182(3-4):143-54.
71. Strachan L R, Scalapino K L, Lawrence H J, Ghadially R. Rapid adhesion to collagen isolates murine keratinocytes with limited long-term repopulating ability in vivo despite high clonogenicity in vitro. Stem Cells. 2007 Oct. 18.
72. Shimaoka M, Takagi J, Springer TA. Conformational regulation of integrin structure and function. Annu Rev Biophys Biomol Struct. 2002; 31:485-516. Review.
73. Xing L, Huhtala M, Pietiäinen V, Käpylä J, Vuorinen K, Marjomäki V, Heino J, Johnson M S, Hyypiä T, Cheng R H. Structural and functional analysis of integrin alpha2I domain interaction with echovirus 1. J Biol Chem. 2004 Mar. 19; 279(12):11632-8.
74, Park H B, Golubovskaya V, Xu L, Yang X, Lee J W, Scully S 2nd, Craven R J, Cance WG. Activated Src increases adhesion, survival and alpha2-integrin expression in human breast cancer cells. Biochem J. 2004 Mar. 1; 378(Pt 2):559-67.
75. Suzuki-Inoue K, Yatomi Y, Asazuma N, Kainoh M, Tanaka T, Satoh K, Ozaki Y. Rac, a small guanosine triphosphate-binding protein, and p21-activated kinase are activated during platelet spreading on collagen-coated surfaces: roles of integrin alpha(2)beta(1). Blood. 2001 Dec. 15; 98(13):3708-16.
76. Avizienyte E, Wyke A W, Jones R J, McLean G W, Westhoff M A, Brunton V G, Frame M C. Src-induced de-regulation of E-cadherin in colon cancer cells requires integrin signalling. Nat Cell Biol. 2002 August; 4(8):632-8.
77. Cheli Y, Kanaji S, Jacquelin B, Chang M, Nugent D J, Kunicki T J. Transcriptional and epigenetic regulation of the integrin collagen receptor locus ITGA1-PELO-ITGA2. Biochim Biophys Acta. 2007 September-October; 1769(9-10):546-58.
78. Cheli Y, Kunicki TJ. hnRNP L regulates differences in expression of mouse integrin alpha2beta1. Blood. 2006 Jun. 1; 107(11):4391-8.
79. Langsenlehner U, Renner W, Yazdani-Biuki B, Eder T, Wascher T C, Paulweber B, Clar H, Hofmann G, Samonigg H, Krippl P. Integrin alpha-2 and beta-3 gene polymorphisms and breast cancer risk. Breast Cancer Res Treat. 2006 May; 97(1):67-72.
80. Di Paola J, Jugessur A, Goldman T, Reiland J, Tallman D, Sayago C, Murray J C. Platelet glycoprotein I(b)alpha and integrin alpha2 beta1 polymorphisms: gene frequencies and linkage disequilibrium in a population diversity panel. J Thromb Haemost. 2005 July; 3(7):1511-21.
81. Block K L, Shou Y, Thorton M, Poncz M. The regulated expression of a TATA-less, platelet-specific gene, alphaIIb. Stem Cells. 1996; 14 Suppl 1:38-47. Review.
82. Jacquelin B, Rozenshteyn D, Kanaji S, Koziol J A, Nurden A T, Kunicki T J. Characterization of Inherited Differences in Transcription of the Human Integrin alpha 2 Gene. J Biol Chem. 2001 Jun. 29; 276(26):23518-24.
83. Jacquelin B, Tarantino M D, Kritzik M, Rozenshteyn D, Koziol J A, Nurden A T, Kunicki T J. Allele-dependent transcriptional regulation of the human integrin alpha2 gene. Blood. 2001 Mar. 15; 97(6):1721-6.
84. Corbi A L, Jensen U B, Watt F M. The alpha2 and alpha5 integrin genes: identification of transcription factors that regulate promoter activity in epidermal keratinocytes. FEBS Lett. 2000 Jun. 2; 474(2-3):201-7.
85. Xiao G, Wang D, Benson M D, Karsenty G, Franceschi R T. Role of the alpha2-integrin in osteoblast-specific gene expression and activation of the Osf2 transcription factor. J Biol Chem. 1998 Dec. 4; 273(49):32988-94.
86. Xu J, Zutter M M, Santoro S A, Clark R A. A three-dimensional collagen lattice activates NF-kappaB in human fibroblasts: role in integrin alpha2 gene expression and tissue remodeling. J Cell Biol. 1998 Feb. 9; 140(3):709-19.
87. Zutter M M, Ryan E E, Painter A D. Binding of phosphorylated Sp1 protein to tandem Sp1 binding sites regulates alpha2 integrin gene core promoter activity. Blood. 1997 Jul. 15; 90(2):678-89.
88. Zutter M M, Santoro S A, Painter A S, Tsung Y L, Gafford A. The human alpha 2 integrin gene promoter. Identification of positive and negative regulatory elements important for cell-type and developmentally restricted gene expression. J Biol Chem. 1994 Jan. 7; 269(1):463-9.
89. Nissinen L, Westermarck J, Koivisto L, Kähäri V M, Heino J. Transcription of alpha2 integrin gene in osteosarcoma cells is enhanced by tumor promoters. Exp Cell Res. 1998 Aug. 25; 243(1):1-10.
90. Zhu Y H, Vekemans S, Cassiman J J. Semiquantitative PCR of alpha2 and alpha4 integrin mRNA shows differential response to the transcriptional modulators TGF-beta1 and TPA. Cell Mol Biol (Noisy-le-grand). 1997 December; 43(8):1221-6.
91. Athanasiou M, Clausen P A, Mavrothalassitis G J, Zhang X K, Watson D K, Blair D G. Increased expression of the ETS-related transcription factor FLI-1/ERGB correlates with and can induce the megakaryocytic phenotype. Cell Growth Differ. 1996 November; 7(11):1525-34.
92. Chen H, Paul WE. A population of CD62Llow Nk1.1− CD4+ T cells that resembles NK1.1+ CD4+ T cells. Eur J. Immunol. 1998 October; 28(10):3172-82.
93. Caminschi I, Ahmet F, Heger K, Brady J, Nutt S L, Vremec D, Pietersz S, Lahoud M H, Schofield L, Hansen DS, O'Keeffe M, Smyth M J, Bedoui S, Davey G M, Villadangos J A, Heath W R, Shortman K. Putative IKDCs are functionally and developmentally similar to natural killer cells, but not to dendritic cells. J Exp Med. 2007 Oct. 29; 204(11):2579-90.
94. Ioachim E, Charchanti A, Briasoulis E, Karavasilis V, Tsanou H, Arvanitis D L, Agnantis N J, Pavlidis N. Immunohistochemical expression of extracellular matrix components tenascin, fibronectin, collagen type IV and laminin in breast cancer: their prognostic value and role in tumour invasion and progression. Eur J. Cancer. 2002 December; 38(18):2362-70.
95, Xie J, Haslam SZ. Extracellular matrix regulates ovarian hormone-dependent proliferation of mouse mammary epithelial cells. Endocrinology. 1997 June; 138(6):2466-73.
96. Laing G D, Moura-da-Silva A M. Jararhagin and its multiple effects on hemostasis. Toxicon. 2005 Jun. 15; 45(8):987-96. Review.
97. Souza D H, lemma M R, Ferreira L L, Faria J P, Oliva M L, Zingali R B, Niewiarowski S, Selistre-de-Araujo HS. The disintegrin-like domain of the snake venom metalloprotease alternagin inhibits alpha2beta1 integrin-mediated cell adhesion. Arch Biochem Biophys. 2000 Dec. 15; 384(2):341-50.
98. De Luca M, Ward C M, Ohmori K, Andrews R K, Berndt MC. Jararhagin and jaracetin: novel snake venom inhibitors of the integrin collagen receptor, alpha2beta1. Biochem Biophys Res Commun. 1995 Jan. 17; 206(2):570-6.
99. Kamiguti A S, Hay C R, Zuzel M. Inhibition of collagen-induced platelet aggregation as the result of cleavage of alpha 2 beta 1-integrin by the snake venom metalloproteinase jararhagin. Biochem J. 1996 Dec. 1; 320 (Pt 2):635-41.
100. Ivaska J, Käpylä J, Pentikäinen O, Hoffrén A M, Hermonen J, Huttunen P, Johnson M S, Heino J. A peptide inhibiting the collagen binding function of integrin alpha2I domain. J Biol Chem. 1999 Feb. 5; 274(6):3513-21.
101. Costa E P, Santos M F. Jararhagin, a snake venom metalloproteinase-disintegrin, stimulates epithelial cell migration in an in vitro restitution model. Toxicon. 2004 Dec. 15; 44(8):861-70.
102. Selistre-de-Araujo H S, Cominetti M R, Terruggi C H, Mariano-Oliveira A, De Freitas M S, Crepin M, Figueiredo C C, Morandi V. Alternagin-C, a disintegrin-like protein from the venom of Bothrops alternatus, modulates alpha2beta1 integrin-mediated cell adhesion, migration and proliferation. Braz J Med Biol Res. 2005 October; 38(10):1505-11. Review.
103. Mariano-Oliveira A, Coelho A L, Terruggi C H, Selistre-de-Araújo HS, Barja-Ficlalgo C, De Freitas M S. Alternagin-C, a nonRGD-disintegrin, induces neutrophil migration via integrin signaling. Eur J. Biochem. 2003 December; 270(24):4799-808.
104. Coelho A L, de Freitas M S, Oliveira-Carvalho A L, Moura-Neto V, Zingali R B, Barja-Fidalgo C. Effects of jarastatin, a novel snake venom disintegrin, on neutrophil migration and actin cytoskeleton dynamics. Exp Cell Res. 1999 Sep. 15; 251(2):379-87.
105. Riikonen R, Matilainen H, Rajala N, Pentikäinen O, Johnson M, Heino J. Oker-Blom C. Functional display of an alpha2 integrin-specific motif (RKK) on the surface of baculovirus particles. Technol Cancer Res Treat. 2005 August; 4(4):437-45.
106. Pentikäinen O, Hoffrén A M, Ivaska J, Käpylä J, Nyrönen T, Heino J, Johnson M S. “RKKH” peptides from the snake venom metalloproteinase of Bothrops jararaca bind near the metal ion-dependent adhesion site of the human integrin alpha(2) I-domain. J Biol Chem. 1999 Oct. 29; 274(44):31493-505.
107. Nymalm Y, Puranen J S, Nyholm TK, Käpylä J, Kidron H, Pentikäinen O T, Airenne T T, Heino J, Slotte J P, Johnson M S, Salminen TA. Jararhagin-derived RKKH peptides induce structural changes in alpha1I domain of human integrin alpha1beta1. J Bial Chem. 2004 Feb. 27; 279(9):7962-70.
108. Souza D H, Selistre-de-Araujo H S, Moura-da-Silva A M, Della-Casa M S, Oliva G, Garratt RC. Crystallization and preliminary X-ray analysis of jararhagin, a metalloproteinase/disintegrin from Bothrops jararaca snake venom. Acta Crystallogr D Biol Crystallogr. 2001 August; 57(Pt 8):1135-7.
109. Moura-da-Silva A M, Marcinkiewicz C, Marcinkiewicz M, Niewiarowski S. Selective recognition of alpha2beta1 integrin by jararhagin, a Metalloproteinase/disintegrin from bBothrops jararaca venom. Thromb Res. 2001 Apr. 15; 102(2):153-9.
110, Tanjoni I, Butera D, Bento L, Della-Casa M S, Marques-Porto R, Takehara H A, Gutiérrez J M, Fernandes I, Moura-da-Silva A M. Snake venom metalloproteinases: structure/function relationships studies using monoclonal antibodies. Toxicon. 2003 December; 42(7):801-8.
111. Cidade D A, Wermelinger L S, Lôbo-Hajdu G, Dávila A M, Bon C, Zingali R B, Albano R M. Molecular diversity of disintegrin-like domains within metalloproteinase precursors of Bothrops jararaca. Toxicon. 2006 October; 48(5):590-9.
112. Kamiguti A S, Markland F S, Zhou Q, Laing G D, Theakston R D, Zuzel M. Proteolytic cleavage of the beta1 subunit of platelet alpha2beta1 integrin by the metalloproteinase jararhagin compromises collagen-stimulated phosphorylation of pp 72. J Biol Chem. 1997 Dec. 19; 272(51):32599-605.
113. Drake S L, Varnum J, Mayo K H, Letourneau P C, Furcht L T, McCarthy J B. Structural features of fibronectin synthetic peptide FN-C/H II, responsible for cell adhesion, neurite extension, and heparan sulfate binding. J Biol Chem, 1993 Jul. 25; 268(21):15859-67.
114. Schilling D, Reid IV JD, Hujer A, Morgan D, Demoll E, Bummer P, Fenstermaker R A, Kaetzel DM. Loop III region of platelet-derived growth factor (PDGF) B-chain mediates binding to PDGF receptors and heparin. Biochem J. 1998 Aug. 1; 333 (Pt 3):637-44.
115. Chung C H, Wu W B, Huang T F. Aggretin, a snake venom-derived endothelial integrin alpha 2 beta 1 agonist,induces angiogenesis via expression of vascular endothelial growth factor. Blood. 2004 Mar. 15; 103(6):2105-13.
116. Eble J A, Beermann B, Hinz H J, Schmidt-Hederich A. alpha 2beta 1 integrin is not recognized by rhodocytin but is the specific, high affinity target of rhodocetin, an RGD-independent disintegrin and potent inhibitor of cell adhesion to collagen. J Biol Chem. 2001 Apr. 13; 276(15):12274-84.
117. Stingl J, Raouf A, Emerman J T, Eaves C J. Epithelial progenitors in the normal human mammary gland. J Mammary Gland Biol Neoplasia. 2005 January; 10(1):49-59. Review.
118. Cariati M, Naderi A, Brown J P, Smalley M J, Pinder S E, Caldas C, Purushotham AD. Alpha-6 integrin is necessary for the tumourigenicity of a stem cell-like subpopulation within the MCF7 breast cancer cell line. Int J. Cancer. 2008 Jan. 15; 122(2):298-304.
119. Yamada Y, Matsuo H, Segawa T, Watanabe S, Kato K, Hibino T, Yokoi K, Ichihara S, Metoki N, Yoshida H, Satoh K, Nozawa Y. Assessment of the genetic component of hypertension. Am J Hypertens. 2006 November; 19(11):1158-65.
120. Matarin M, Brown W M, Hardy J A, Rich S S, Singleton A B, Brown R D Jr, Brott T G, Worrall B B, Meschia J F. Association of integrin alpha2 gene variants with ischemic stroke. J Cereb Blood Flow Metab. 2008 January; 28(1):81-9.
121. Kunicki T J, Baronciani L, Canciani M T, Gianniello F, Head S R, Mondala T S, Salomon D R, Federici A B. An association of candidate gene haplotypes and bleeding severity in von Willebrand disease type 2A, 2B, and 2M pedigrees. J Thromb Haemost. 2006 January; 4(1):137-47.
122. Asselah T, Bieche I, Laurendeau I, Paradis V, Vidaud D, Degott C, Martinot M, Bedossa P, Valla D, Vidaud M, Marcellin P. Liver gene expression signature of mild fibrosis in patients with chronic hepatitis C. Gastroenterology. 2005 December; 129(6):2064-75.
123. Charbonnier LM, van Duivenvoorde L M, Apparailly F, Cantos C, Han W G, Noël D, Duperray C, Huizing a T W, Toes R E, Jorgensen C, Louis-Plence P. Immature dendritic cells suppress collagen-induced arthritis by in vivo expansion of CD49b+ regulatory T cells. J. Immunol. 2006 Sep. 15; 177(6):3806-13.
124. Kassiotis G, Gray D, Kiafard Z, Zwirner J, Stockinger B. Functional specialization of memory Th cells revealed by expression of integrin CD49b. J Immunol. 2006 Jul. 15; 177(2):968-75.
125. Edelson B T, Li Z, Pappan L K, Zutter M M. Mast cell-mediated inflammatory responses require the alpha 2 beta 1 integrin. Blood.
2004 Mar. 15; 103(6):2214-20.
126. Zutter M M, Edelson B T, The alpha2beta1 integrin: a novel collectin/C1q receptor. Immunobiology. 2007; 212(4-5):343-53. Review.
127. Graham K L, Zeng W, Takada Y, Jackson D C, Coulson BS. Effects on rotavirus cell binding and infection of monomeric and polymeric peptides containing alpha2beta1 and alphaxbeta2 integrin ligand sequences. J. Virol. 2004 November; 78(21):11786-97.
128. Sweeney S M, DiLullo G, Slater S J, Martinez J, Iozzo R V, Lauer-Fields J L, Fields G B, San Antonio J D. Angiogenesis in collagen I requires alpha2beta1 ligation of a GFP*GER sequence and possibly p38 MAPK activation and focal adhesion disassembly. J Biol Chem, 2003 Aug. 15; 278(33):30516-24.
129. Sanz L, García-Bermejo L, Blanco F J, Kristensen P, Feijóo M, Suarez E, Blanco B, Alvarez-Vallina L. A novel cell binding site in the coiled-coil domain of laminin involved in capillary morphogenesis. EMBO J. 2003 Apr. 1; 22(7):1508-17.
130. Bix G, Castello R, Burrows M, Zoeller J J, Weech M, Iozzo R A, Cardi C, Thakur M L, Barker C A, Camphausen K, Iozzo RV. Endorepellin in vivo: targeting the tumor vasculature and retarding cancer, growth and metabolism. J Natl Cancer Inst. 2006 Nov. 15; 98(22):1634-46.
131. Alghisi G C, Rüegg C. Vascular integrins in tumor angiogenesis: mediators and therapeutic targets. Endothelium. 2006 March-April; 13(2):113-35. Review.
132. Parks W C. What is the alpha2beta1 integrin doing in the epidermis?J Invest Dermatol. 2007 February; 127(2):264-6. Review.
133. Semba T, Funahashi Y, Ono N, Yamamoto Y, Sugi N H, Asada M, Yoshimatsu K, Wakabayashi T. An angiogenesis inhibitor E7820 shows broad-spectrum tumor growth inhibition in a xenograft model: possible value of integrin alpha2 on platelets as a biological marker. Clin Cancer Res. 2004 Feb. 15; 10(4):1430-8.
134. Woodall B P, Nystrom A, Iozzo R A, Eble J A, Niland S, Krieg T, Eckes B, Pozzi A, Iozzo RV. Integrin alpha 2beta 1 is the required receptor for endorepellin angiostatic activity. J Biol Chem. 2008 Jan. 25; 283(4):2335-43.
135. Li T T, Larrucea 5, Souza S, Leal S M, López J A, Rubin E M, Nieswandt B, Bray P F. Genetic variation responsible for mouse strain differences in integrin alpha 2 expression is associated with altered platelet responses to collagen. Blood. 2004 May 1; 103(9):3396-402.
136. Cines D B, Blanchette VS. Immune thrombocytopenic purpura. N Engl J. Med. 2002 Mar. 28; 346(13):995-1008. Review.
137. Matsubara Y, Murata M, Maruyama T, Handa M, Yamagata N, Watanabe G, Saruta T, Ikeda Y. Association between diabetic retinopathy and genetic variations in alpha2beta1 integrin, a platelet receptor for collagen. Blood. 2000 Mar. 1; 95(5):1560-4.
138. Zutter M M, Sun H, Santoro S A. Altered integrin expression and the malignant phenotype: the contribution of multiple integrated integrin receptors, J Mammary Gland Biol Neoplasia. 1998 April; 3(2):191-200. Review.
139. Pec M K, Artwohl M, Fernández J J, Souto M L, Alvarez de la Rosa D, Giraldez T, Valenzuela-Fernández A, Díaz-González F. Chemical modulation of VLA integrin affinity in human breast cancer cells. Exp Cell Res. 2007 Apr. 1; 313(6):1121-34.
140. Hartstein M E, Grove A S Jr, Woog J J. The role of the integrin family of adhesion molecules in the development of tumors metastatic to the orbit. Ophthal Plast Reconstr Surg. 1997 December; 13(4):227-38.
141. Lang K, Drell T L 4th, Lindecke A, Niggemann B, Kaltschmidt C, Zaenker K S, Entschladen F. Induction of a metastatogenic tumor cell type by neurotransmitters and its pharmacological inhibition by established drugs. Int J. Cancer. 2004 Nov. 1; 112(2):231-8.
142, Zutter M M, Santoro S A, Staatz W D, Tsung Y L. Re-expression of the alpha 2 beta 1 integrin abrogates the malignant phenotype of breast carcinoma cells. Proc Nati Acad Sci USA. 1995 Aug. 1; 92(16):7411-5.
143. Lochter A, Navre M, Werb Z, Bissell M J. alpha1 and alpha2 integrins mediate invasive activity of mouse mammary carcinoma cells through regulation of stromelysin-1 expression. Mol Biol Cell. 1999 February; 10(2):271-82.
144. Connors W L, Jokinen J, White D J, Puranen J S, Kankaanpää P, Upla P, Tulla M, Johnson M S, Heino J. Two synergistic activation mechanisms of alpha2beta1 integrin-mediated collagen binding. J Biol Chem. 2007 May 11; 282(19):14675-83.
145. Schneider G B, Zaharias R, Stanford C. Osteoblast integrin adhesion and signaling regulate mineralization. J Dent Res. 2001 June; 80(6):1540-4.
146. Lundström A, Holmbom J, Lindqvist C, Nordström T. The role of alpha2 beta1 and alpha beta1 integrin receptors in the initial anchoring of MDA-MB-231 human breast cancer cells to cortical bone matrix. Biochem Biophys Res Commun. 1998 Sep. 29; 250(3):735-40.
147. Judware R, Culp L A. N-myc over-expression down-regulates alpha3beta1 integrin expression in human Saos-2 osteosarcoma cells. Clin Exp Metastasis. 1997 May; 15(3):228-38.
148. Kostenuik P J, Singh G, Orr F W. Transforming growth factor beta upregulates the integrin-mediated adhesion of human prostatic carcinoma cells to type I collagen. Clin Exp Metastasis. 1997 January; 15(1):41-52.
149. Vihinen P, Riikonen T, Laine A, Heino J. Integrin alpha 2 beta 1 in tumorigenic human osteosarcoma cell lines regulates cell adhesion, migration, and invasion by interaction with type I collagen. Cell Growth Differ. 1996 April; 7(4):439-47.
150. Hikawa T, Mori T, Abe T, Hori S. The ability in adhesion and invasion of drug-resistant human glioma cells. J Exp Clin Cancer Res. 2000 September; 19(3):357-62.
151. Morozevich G E, Kozlova N I, Preobrazhenskaya M E, Ushakova N A, Eltsov I A, Shtil A A, Berman A E. The role of beta1 integrin subfamily in anchorage-dependent apoptosis of breast carcinoma cells differing in multidrug resistance. Biochemistry (Mosc). 2006 May; 71(5):489-95.
152. Lang K, Drell TL 4th, Lindecke A, Niggemann B, Kaltschmidt C, Zaenker K S, Entschladen F. Induction of a metastatogenic tumor cell type by neurotransmitters and its pharmacological inhibition by established chugs. Int J. Cancer. 2004 Nov. 1; 112(2):231-8.
153. Baeckström D, Lu P J, Taylor-Papadimitriou J. Activation of the alpha2beta1 integrin prevents c-erbB2-induced scattering and apoptosis of human mammary epithelial cells in collagen. Oncogene. 2000 Sep. 21; 19(40):4592-603.
154. Xu J, Clark R A. Extracellular matrix alters PDGF regulation of fibroblast integrins. J Cell Biol. 1996 January; 132(1-2):239-49.
155. Ahlén K, Rubin K. Platelet-derived growth factor-BB stimulates synthesis of the integrin alpha 2-subunit in human diploid fibroblasts. Exp Cell Res. 1994 December; 215(2):347-53.
156. Hallenbeck S T, Itoh H, Louie O, Faries P L, Liu B, Kent K C. Type I collagen synergistically enhances PDGF-induced smooth muscle cell proliferation through pp 60src-dependent crosstalk between the alpha2beta1 integrin and PDGFbeta receptor. Biochem Biophys Res Commun. 2004 Dec. 3; 325(1):328-37.
157. Grundström G, Mosher D F, Sakai T, Rubin K. Integrin alphavbeta3 mediates platelet-derived growth factor-BB-stimulated collagen gel contraction in cells expressing signaling deficient integrin alpha2beta1. Exp Cell Res. 2003 Dec. 10; 291(2):463-73.
158. Xu J, Zutter M M, Santoro S A, Clark R A. PDGF induction of alpha 2 integrin gene expression is mediated by protein kinase C-zeta. J Cell Biol. 1996 September; 134(5):1301-11.
159. Bando H, Ikuno Y, Hori Y, Sayanagi K, Tano Y. Mitogen-activated protein kinase (MAPK) and phosphatidylinositol-3 kinase (PI3K) pathways differently regulate retinal pigment epithelial cell-mediated collagen gel contraction. Exp Eye Res. 2006 March; 82(3):529-37.
160. Whittard J D, Craig S E, Mould A P, Koch A, Pertz O, Engel J, Humphries M J. E-cadherin is a ligand for integrin alpha2beta1. Matrix Biol. 2002 October; 21(6):525-32.
161. André F, Rigot V, Thimonier J, Montixi C, Parat F, Pommier G, Marvaldi J, Luis J. Integrins and E-cadherin cooperate with IGF-I to induce migration of epithelial colonic cells. Int J. Cancer. 1999 Nov. 12; 83(4):497-505.
162. Grzesiak J J, Pierschbacher M D. Changes in the concentrations of extracellular Mg++ and Ca++ down-regulate E-cadherin and up-regulate alpha 2 beta 1 integrin function, activating keratinocyte migration on type I collagen. J Invest Dermatol. 1995 May; 104(5):768-74.
163. Yamaguchi K, Ura H, Yasoshima T, Shishido T, Denno R, Hirata K. Establishment and characterization of a human gastric carcinoma cell line that is highly metastatic to lymph nodes. J Exp Clin Cancer Res. 2000 March; 19(1):113-20.
164. Dahlman T, Grimelius L, Wallin G, Rubin K, Westermark K. Integrins in thyroid tissue: upregulation of alpha2beta1 in anaplastic thyroid carcinoma. Eur J. Endocrinol. 1998 January; 138(1):104-12.
165. Aquilina A, Korda M, Bergelson J M, Humphries M J, Farndale R W, Tuckwell D. A novel gain-of-function mutation of the integrin alpha2 VWFA domain. Eur J. Biochem. 2002 February; 269(4):1136-44.
166. Tuckwell D S, Smith L, Korda M, Askari J A, Santoso S, Barnes M J, Farndale R W, Humphries M J. Monoclonal antibodies identify residues 199-216 of the integrin alpha2 vWFA domain as a functionally important region within alpha2beta1. Biochem J. 2000 Sep. 1; 350 Pt 2:485-93.
167. Zhang W, Alt-Holland A, Margulis A, Shamis Y, Fusenig N E, Rodeck U, Garlick J A. E-cadherin loss promotes the initiation of squamous cell carcinoma invasion through modulation of integrin-mediated adhesion. J Cell Sci. 2006 Jan. 15; 119(Pt 2):283-91.
168. Maschler S, Wirl G, Spring H, Bredow D V, Sordat I, Beug H, Reichmann E. Tumor cell invasiveness correlates with changes in integrin expression and localization. Oncogene. 2005 Mar. 17; 24(12):2032-41.
169. Zoppi N, Gardella R, De Paepe A, Barlati S, Colombi M. Human fibroblasts with mutations in COL5A1 and COL3A1 genes do not organize collagens and fibronectin in the extracellular matrix, down-regulate alpha2beta1 integrin, and recruit alphavbeta3 Instead of alpha5beta1 integrin. J Biol Chem. 2004 Apr. 30; 279(18):18157-68.
170. Byzova T V, Goldman C K, Pampori N, Thomas K A, Bett A, Shattil SJ, Plow E F. A mechanism for modulation of cellular responses to VEGF: activation of the integrins. Mol Cell. 2000 October; 6(4):851-60.
171. Peng Q, Lai D, Nguyen T T, Chan V, Matsuda T, Hirst 53. Multiple beta 1 integrins mediate enhancement of human airway smooth muscle cytokine secretion by fibronectin and type I collagen. J Immunol. 2005 Feb. 15; 174(4):2258-64.
172. Barazi H O, Li Z, Cashel J A, Krutzsch H C, Annis D S, Mosher D F, Roberts DD. Regulation of integrin function by CD47 ligands. Differential effects on alphavbeta 3 and alpha 4beta1 integrin-mediated adhesion. J Biol Chem. 2002 Nov. 8; 277(45):42859-66.
173. Halasz P, Holloway G, Turner S J, Coulson BS. Rotavirus replication in intestinal cells differentially regulates integrin expression by a phosphatidylinositol 3-kinase-dependent pathway, resulting in increased cell adhesion and virus yield. J. Virol. 2008 January; 82(1):148-60.
174. Frisch S M, Screaton R A. Anoikis mechanisms. Curr Opin Cell Biol. 2001 October; 13(5):555-62. Review.
175. Martin S S, Vuori K. Regulation of Bcl-2 proteins during anoikis and amorphosis. Biochim Biophys Acta. 2004 Jul. 5; 1692(2-3):145-57. Review.
176. Gendron S, Couture J, Aoudjit F. Integrin alpha2beta1 inhibits Fas-mediated apoptosis in T lymphocytes by protein phosphatase 2A-dependent activation of the MAPK/ERK pathway. J Biol Chem. 2003 Dec. 5; 278(49):48633-43.
177. Conant K, St Hillaire C, Nagase H, Visse R, Gary D, Haughey N, Anderson C, Turchan J, Nath A. Matrix metalloproteinase 1 interacts with neuronal integrins and stimulates dephosphorylation of Akt. J Biol Chem. 2004 Feb. 27; 279(9):8056-62.
178. Park K J, Lee S H, Kim T I, Lee H W, Lee C H, Kim E H, Jang H J, Choi K S, Kwon M H, Kim Y S. A human scFv antibody against TRAIL receptor 2 induces autophagic cell death in both TRAIL-sensitive and TRAIL-resistant cancer cells. Cancer Res. 2007 Aug. 1; 67(15):7327-34.
179. Thorburn J, Moore F, Rao A, Barclay W W, Thomas L R, Grant K W, Cramer S D, Thorburn A. Selective inactivation of a Fas-associated death domain protein (FADD)-dependent apoptosis and autophagy pathway in immortal epithelial cells. Mol Biol Cell. 2005 March; 16(3):1189-99.
180. Mills K R, Reginato M, Debnath J, Queenan B, Brugge J S. Tumor necrosis factor-related apoptosis-inducing ligand (TRAIL) is required for induction of autophagy during lumen formation in vitro. Proc Nati Acad Sci USA. 2004 Mar. 9; 101(10):3438-43.
181. Taieb J, Chaput N, Ménard C, Apetoh L, Ullrich E, Bonmort M, Péquignot M, Casares N, Terme M, Flament C, Opolon P, Lecluse Y, Métivier D, Tomasello E, Vivier E, Ghiringhelli F, Martin F, Klatzmann D, Poynard T, Tursz T, Raposo G, Yagita H, Ryffel B, Kroemer G, Zitvogel L. A novel dendritic cell subset involved in tumor immunosurveillance. Nat Med. 2006 February; 12(2):214-9.
182. Prince J M, Klinowska T C, Marshman E, Lowe E T, Mayer U, Miner J, Aberdam D, Vestweber D, Gusterson B, Streuli C H. Cell-matrix interactions during development and apoptosis of the mouse mammary gland in vivo. Dev Dyn. 2002 April; 223(4):497-516.
183. Laplante P, Raymond M A, Labelle A, Abe J, Iozzo R V, Hebert M J. Perlecan proteolysis induces an alpha2beta1 integrin- and Src family kinase-dependent anti-apoptotic pathway in fibroblasts in the absence of focal adhesion kinase activation. J Biol Chem. 2006 Oct. 13; 281(41):30383-92.
184. Rubinsztein D C, Gestwicki J E, Murphy L O, Klionsky D J. Potential therapeutic applications of autophagy. Nat Rev Drug Discov. 2007 April; 6(4):304-12. Review.
185. Easton J B, Houghton PJ. mTOR and cancer therapy. Oncogene. 2006 Oct. 16; 25(48):6436-46. Review.
186. Sarbassov D D, Ali S M, Sabatini D M. Growing roles for the mTOR pathway. Curr Opin Cell Biol. 2005 December; 17(6):596-603. Epub 2005 Oct. 13. Review.
187. Lum J J, DeBerardinis R J, Thompson CB. Autophagy in metazoans: cell survival in the land of plenty. Nat Rev Mol Cell Biol. 2005 June; 6(6):439-48. Review.
188. Neshat M S, Mellinghoff I K, Tran C, Stiles B, Thomas G, Petersen R, Frost P, Gibbons J J, Wu H, Sawyers C L. Enhanced sensitivity of PTEN-deficient tumors to inhibition of FRAP/mTOR. Proc Natl Acad Sci USA. 2001 Aug. 28; 98(18):10314-9.
189. Podsypanina K, Lee R T, Politis C, Hennessy I, Crane A, Puc J, Neshat M, Wang H, Yang L, Gibbons J, Frost P, Dreisbach V, Blenis J, Gaciong Z, Fisher P, Sawyers C, Hedrick-Ellenson L, Parsons R. An inhibitor of mTOR reduces neoplasia and normalizes p70/S6 kinase activity in Pten+/− mice. Proc Natl Acad Sci USA. 2001 Aug. 28; 98(18): 10320-5.
190. Laun P, Bruschi C V, Dickinson J R, Rinnerthaler M, Heeren G, Schwimbersky R, Rid R, Breitenbach M. Yeast mother cell-specific ageing, genetic (in)stability, and the somatic mutation theory of ageing. Nucleic Acids Res. 2007; 35(22):7514-26.
191. Jin J, Woodgett JR. Chronic activation of protein kinase Bbeta/Akt2 leads to multinucleation and cell fusion in human epithelial kidney cells: events associated with tumorigenesis. Oncogene. 2005 Aug. 18; 24(35):5459-70.
192. Bras M, Queenan B, Susin S A. Programmed cell death via mitochondria: different modes of dying. Biochemistry (Mosc). 2005 February; 70(2):231-9. Review.
193. Mellor H R, Harris A L. The role of the hypoxia-inducible BH3-only proteins BNIP3 and BNIP3L in cancer, Cancer Metastasis Rev. 2007 December; 26(3-4):553-66. Review.
194. Sowter H M, Ferguson M, Pym C, Watson P, Fox S B, Han C, Harris A L. Expression of the cell death genes BNip3 and NIX in ductal carcinoma in situ of the breast; correlation of BNip3 levels with necrosis and grade. J Pathol. 2003 December; 201(4):573-80.
195. Maiuri M C, Criollo A, Tasdemir E, Vicencio J M, Tajeddine N, Hickman J A, Geneste O, Kroemer G. BH3-only proteins and BH3 mimetics induce autophagy by competitively disrupting the interaction between Beclin 1 and Bcl-2/Bcl-X(L). Autophagy. 2007 July-August; 3(4):374-6.
196. Maiuri M C, Le Toumelin G, Criollo A, Rain J C, Gautier F, Juin P, Tasdemir E, Pierron G, Troulinaki K, Tavernarakis N, Hickman J A, Geneste O, Kroemer G. Functional and physical interaction between Bcl-X(L) and a BH3-like domain in Beclin-1. EMBO J. 2007 May 16; 26(10):2527-39.
197. Neshat M S, Raitano A B, Wang H G, Reed J C, Sawyers C L. The survival function of the Bcr-Abl oncogene is mediated by Bad-dependent and -independent pathways: roles for phosphatidylinositol 3-kinase and Raf. Mol Cell Biol. 2000 February; 20(4):1179-86.
198. Hewer-Hansen M, Bastholm L, Szyniarowski P, Campanella M, Szabadkai G, Farkas T, Bianchi K, Fehrenbacher N, Elling F, Rizzuto R, Mathiasen IS, Jäättelä M. Control of macroautophagy by calcium, calmodulin-dependent kinase kinase-beta, and Bcl-2. Mol Cell. 2007 Jan. 26; 25(2):193-205.
199. Demasters G, Di X, Newsham I, Shiu R, Gewirtz DA. Potentiation of radiation sensitivity in breast tumor cells by the vitamin D3 analogue, EB 1089, through promotion of autophagy and interference with proliferative recovery. Mol Cancer Ther. 2006 November; 5(11):2786-97.
200. vom Dahl. 5, Schliess F, Reissmann R, Görg B, Weiergräber O, Kocalkova M, Dombrowski F, Haussinger D. Involvement of integrins in osmosensing and signaling toward autophagic proteolysis in rat liver. J Biol Chem. 2003 Jul. 18; 278(29):27088-95.
200. Barrett T, Brechbiel M, Bernardo M, Choyke P L. MRI of tumor angiogenesis. J Magn Reson Imaging. 2007 August; 26(2):235-49. Review.
201. Meairs S, Hennerici M. Future developments in neurovascular ultrasound. Front Neural Neurosci. 2006; 21:261-8. Review.
202. Cai W, Rao J, Gambhir S S, Chen X. How molecular imaging is speeding up antiangiogenic drug development. Mol Cancer Ther. 2006 November; 5(11):2624-33. Review.
203. Chen X, Park R, Khankaldyyan V, Gonzales-Gomez I, Tohme M, Moats R A, Bading J R, Laug W E, Conti P S. Longitudinal microPET imaging of brain tumor growth with F-18-labeled RGD peptide. Mol Imaging Biol. 2006 January-February; 8(1):9-15.
204. Mouchess M L, Sahara Y, Nelson M D Jr, DeCLerck Y A, Moats R A. Multimodal imaging analysis of tumor progression and bone resorption in a murine cancer model. J Comput Assist Tomogr. 2006 May-June; 30(3):525-34.
205. Liao C P, Zhong C, Saribekyan G, Bading J, Park R, Conti P S, Moats R, Berns A, Shi W, Zhou Z, Nikitin A Y, Roy-Burman P. Mouse models of prostate adenocarcinoma with the capacity to monitor spontaneous carcinogenesis by bioluminescence or fluorescence. Cancer Res. 2007 Aug. 1; 67(15):7525-33.
206. Swenson S, Costa F, Ernst W, Fujii G, Markland F S. Contortrostatin, a snake venom disintegrin with anti-angiogenic and anti-tumor activity. Pathophysiol Haemost Thromb. 2005; 34(4-5):169-76.
207. Swenson S, Costa F, Minea R, Sherwin R P, Ernst W, Fujii G, Yang D, Markland F S Jr. Intravenous liposomal delivery of the snake venom disintegrin contortrostatin limits breast cancer progression. Mol Cancer Ther. 2004 April; 3(4):499-511.
208. Minea R, Swenson S, Costa F, Chen T C, Markland F S. Development of a novel recombinant disintegrin, contortrostatin, as an effective anti-tumor and anti-angiogenic agent. Pathophysiol Haemost Thromb. 2005; 34(4-5):177-83.
209. Swenson S, Ramu S, Markland F S. Anti-angiogenesis and RG D-containing snake venom disintegrins. Curr Pharm Des. 2007; 13(28):2860-71. Review.
210. Markland F S. Snake venoms and the hemostatic system. Toxicon.
1998 December; 36(12):1749-800. Review.

All publications cited herein are incorporated by reference in their entirety.

1-41. (canceled)

42. A peptide comprising the sequence of KAHKKRAD (SEQ ID NO:1) or KARKKHAD (SEQ ID NO:2), wherein the length of the peptide is in the range of 8-50 amino acids.

43. The peptide of claim 42, wherein the length of the peptide is in the range of 8-20 amino acids.

44. The peptide of claim 43, wherein the length of the peptide is in the range of 8-12 amino acids.

45. The peptide of claim 42, wherein the peptide is cyclic.

46. The peptide of claim 45, wherein the peptide is cyclized via a link between a side chain and the backbone.

47. The peptide of claim 45 wherein the peptide is cyclized via a link between two reactive groups on the backbone.

48. The peptide of claim 42, wherein the peptide is detectably labeled.

49. The peptide of claim 42, wherein the peptide is linked to an imaging or therapeutic agent.

50. The peptide of claim 42, wherein the peptide is cyclized via a link between the side chain of D and the backbone of K.

51. A composition comprising a pharmaceutically acceptable carrier and the peptide of claim 42.

52. A method of binding the peptide of claim 42 to an aI domain, comprising contacting the peptide of claim 42 with an aI domain, thereby allowing binding of the peptide to the aI domain.

53. The method of claim 52, wherein the aI domain is in α₂, α₁, α₁₀, or α₁₁.

54. The method of claim 52, wherein the aI domain is in α₂β₁.

55. The method of claim 52, wherein the aI domain is on or in a cell.

56. The method of claim 55, wherein the cell is a cancer cell.

57. The method of claim 56, wherein the cancer is breast or ovarian cancer.

58. The method of claim 55, wherein the cell is in a subject.

59. The method of claim 58, wherein the subject is mouse.

60. A method of detecting cells expressing an aI domain, comprising:
contacting the peptide of claim 42 with a cell; and
detecting binding of the peptide to an aI domain on or in the cell.

61. The method of claim 60, wherein the cell is a cancer cell.

62. The method of claim 61, wherein the cancer is breast or ovarian cancer.

63. The method of claim 60, wherein the cell is in a subject.

64. The method of claim 63, wherein the subject is mouse.

65. The method of claim 63, further comprising isolating the cell from the subject.

66. The method of claim 60, wherein the binding of the peptide to the aI domain is detected by imaging.

67. A method of monitoring cancer status in a subject, comprising:
introducing cancer cells into a subject;
allowing the cancer to progress at the primary site or to metastasis in the subject;
administering the peptide of claim 42 to the subject; and
detecting the peptide on or in the cancer cells, thereby monitoring the status of the cancer in the subject.

68. The method of claim 67, wherein the subject is mouse.

69. The method of claim 67, wherein the cancer is breast or ovarian cancer.

70. The method of claim 67, wherein the peptide is detected by imaging.
